(19) United States
(12) Patent Application Publication
(10) Pub. No.: US 2006/0165689 A1
(43) Pub. Date: Jul. 27, 2006

(54) Compositions and methods for modulation of effects on phagocyte and lymphoid cell populations employing tirc7

(76) Inventor: Nalan Utku, Berlin (DE)

Correspondence Address: COOPER & DUNHAM, LLP, 1185 AVENUE OF THE AMERICAS, NEW YORK, NY, 10036, US

(21) Appl. No.: 10/514,053

(22) PCT Filed: Feb. 4, 2003
(86) PCT No.: PCT/EP03/01083

§ 371 Date: Jun. 24, 2005

(30) Foreign Application Priority Data
Feb. 4, 2002 (EP) 02001980.8

(51) Int. Cl. A61K 48/00; A61K 39/395

(52) U.S. Cl. 424/144.1; 514/12; 514/44

(57) ABSTRACT

Provided are compositions and methods for the prevention and treatment of mammalian disorders that are ameliorated by modulation of effects on phagocyte and lymphoid cell populations and T-cell immune response cDNA 7 (TIRC7) activity in certain cells. Furthermore, improved methods for the production of immunoglobulins to a desired antigen are described. This invention is based on the discovery of a mechanism for the regulation of phagocytosis and the response of lymphoid cell populations to antigens.

### FIELD OF THE INVENTION

This invention relates to the prevention and treatment of mammalian disorders that are ameliorated by modulation of effects on phagocyte and lymphoid cell populations and T-cell immune response cDNA 7 (TIRC7) activity in certain cells. The invention provides numerous compositions, methods and articles of manufacture, and addresses a considerable range of disorders such as those of skin and the immune and central nervous systems. Furthermore, the invention provides improved methods for the production of immunoglobulins to a desired antigen. This invention is based on the discovery of a mechanism for the regulation of phagocytosis and the response of lymphoid cell populations to antigens.

### BACKGROUND OF THE INVENTION

There are three main categories of white blood cells, granulocytes, monocytes and lymphocytes. Granulocytes all contain numerous lysosomes and secretory vesicles and are subdivided in neutrophils, eosinophils and basophils. Monocytes become tissue macrophages, which phagocytose and digest invading microorganisms and foreign bodies as well as damaged and senescent cells.

Phagocytosis is the cellular process of ingestion, and usually of isolation or destruction, of particulate material. In vertebrates, it is a characteristic function of various leukocytes and reticuloendothelial cells. Phagocytosis serves as an important bodily defense mechanism against infection by microorganisms, and against occlusion of mucous surfaces and tissues by foreign particles and tissue debris. Phagocytosis is distinct from pinocytosis, which is the uptake of fluid by a cell through invagination and formation of vesicles off the plasma membrane. Herein, the terms “phagocytosis” and “cellular ingestion” are used interchangeably. The level of phagocytosis in different cells have important implications.

Numerous examples of these implications are provided here:

Immune-Related and Inflammatory Disorders. The primary cause of pulmonary emphysema is the accumulation of foreign material (e.g. smoke condensate) in the lung. This accumulation is followed by the recruitment of neutrophils that are degranulated during attempted phagocytosis (Ravis, Am. J. Respir. Crit. Care Med. 150 (1994), 5143-5146). Immunological lung disorders such as allergic bronchopulmonary aspergillosis cause mucus plugging of airways, eosinophylic pneumonia and bronchiolitis obliterans. In such diseases, neutrophil elastase cleaved immunoglobulins and digested C3b receptors limit the phagocytosis of pathogens (Greenberger, JAMA, Vol. 278, No. 22, 1997). The increase in neutrophil elastase, while impairing phagocytosis, is beneficial for fighting persistent bacterial infections in the lungs, especially in CF patients (Doring, Am. J. Respir. Crit. Care Med. 150: 6 Pt 2, (1994), 114-117).

Periodontal diseases start with the accumulation of plaque at the base of the teeth, followed by the growth of opportunistic bacteria below the gum line. As with the immune response in emphysema, neutrophils are recruited to the infected site, followed by their degranulation during failed phagocytosis (Travis, Am. J. Respir. Crit. Care Med. Vol. 150 (1994), 5143-5146). The rates of adhesion and ingestion of opsonized Staphylococcus Aureus by polymorphonuclear cells (“PMN's”) from periodontal patients is significantly reduced relative to healthy controls (MacFarlane, J. Periodontol 63 (1992), 908-913).

Individuals who are genetically immuno-compromised, who have acquired immuno-suppression (such as HIV infected individuals), or who have temporarily acquired immuno-suppression (such as that following organ transplantation, foreign implants, valve replacement or cancer treatment, and the like), often suffer from secondary infections.

Pulmonary polymorphnuclear leukocytes from diabetic patients were shown to have reduced phagocytic activities, both at the level of ingestion and killing of bacteria, compared to healthy individuals (e.g. Musclow, Cytobios 65 (1991), medline 15-24). In particular, diabetic abnormalities in the immune response include impaired chemotaxis, impaired phagocytosis and impaired adhesion (Grant-Theule, Periodontal Abstracts 44 (1996), No. 3). These patients often suffer from infections.

Cardiovascular System Disorders. The formation of atherosclerotic plaques is induced by aging or by restenosis following balloon angioplasty. Atherosclerotic lesions contain cholesterol-rich particles, many of which aggregate and are internalized in an unregulated fashion by macrophage phagocytosis. This phagocytic process is independent of the LDL or scavenger receptor. The lipid-loaded macrophages, called foamy cells, can lead to further growth of the atherosclerotic plaque (Hoff, European Heart Journal, II (Supp. E) (1990), 105-115; Robert, Annals New York Acad. of Sciences, 673 (1992), 331-341).

Central Nervous System Disorders. Microglial cells found at the periphery of amyloid plaque cores have been shown to contain plaque fibrils of beta/A4 amyloid (El Hachimi and Foncin, C. R. Acad. Sci. Paris, Sciences de la vie/Life sciences, 317 (1994), 445-451). The ability of microglial cells to phagocytose and clear senile plaque cores is suppressed in the presence of an astrocyte-secreted diffusable factor. This factor prevents the clearance of senile plaques, allowing them to persist in Alzheimer's disease and other neuropathological degenerative processes (DeWitt, Experimental Neurology 149 (1998), 329-340). Neutrophil phagocytosis was found to be reduced in clinically depressed patients. Patients with phobic disorders have reduced phagocytosis and cell-killing capacities. Benzodiazepine compounds, used in the treatment of neurological disorders, were shown to reduce or inhibit phagocytosis (e.g. Covelli, Immunopharmacology and Immunotoxicology, 11 (1989),701-714).

Skin Disorders. Mid-dennal elastosis, a skin disorder, is clinically characterized by the appearance of wrinkles and aged appearance which results, in part, from phagocytosis of morphologically normal elastic tissue (e. g. Fimiani, Arch. Dermatol. Res. 287 (1995), 152-157). Many types of pigmentation disorders exist in diverse forms. These can be inherited (e.g. vitiligo), acquired (e.g. post-inflammatory pityriasis alba, idiophatic guttate hypomelanosis, melasma), and transmitted through infection (e.g. tinea versicolor). These disorders can be benign and self-limiting (e.g. isolated cafe au lait spots, photocontact dermatitis), or a sign of a more serious underlying disease (e.g. multiple cafe au lait spots, malignant acanthosis nigricans) (Hacker, Postgrad Med. 99 (1996), 177-186). Acne vulgaris is a multi-stage disorder. The basic acne lesion is the comedo. The second, inflammatory stage when neutrophils are recruited to the comedo area is the reason the disease progresses. Nearly all problems associated with acne result from this inflammatory phase.

Furthermore, there are two main classes of lymphocytes, both involved in immune responses. B lymphocytes make antibodies, while T lymphocytes kill virus-infected cells and regulate the activities of other white blood cells. The latter are called helper T cells of which there exist two types, T_{H}1 cells, which activate macrophages to destroy microorganisms that they have ingested, and T_{H}2 cells, which stimulate B cells to proliferate and secrete antibodies. The importance of phagocytosis in the treatmet of diseases has been discussed before. Besides their role in natural immune response of the body antibodies and antibody producing cells with various immunospecificities are desirable for therapeutic and diagnostic use, in particular monoclonal antibodies. Antibodies intended for therapeutic and diagnostic use can be problematic and/or laborious to generate because not every antigen is a suitable immunogen such that, for example, monoclonal antibody producing cells can be obtained. The availability of nonhuman transgenic animals, that are immunogen responsive or adjuvants for use as co-immunostimulatory molecules may make possible the convenient production of antibodies against any desired antigen. Furthermore, such adjuvants may be used in vaccines in order to enhance the immune response in the human body to a foreign antigen.

Hence, there is always a need of alternative and improved means and methods for regulating cell-mediated immune responses and antibody responses.

### SUMMARY OF THE INVENTION

This invention provides compositions of matter for treating and preventing certain mammalian disorders.

These compositions, are based on the discovery of a mechanism for the regulation of phagocytosis and the response of lymphoid cell populations to antigens.

In a first aspect, the present invention relates to a composition of matter for treating: therapeutically or prophylacticly a mammal afflicted with a disorder ameliorated by an increase in phagocytosis and/or monocyte population, which comprises a therapeutically effective amount of T-cell immune response cDNA 7 (TIRC7), an activator of TIRC7 or of a nucleic acid molecule encoding said TIRC7 or said activator, and optionally a pharmaceutically or cosmetically acceptable carrier.

In a related aspect, the present invention relates to a composition of matter for treating therapeutically or prophylacticly a mammal afflicted with a disorder ameliorated by a decrease in phagocytosis and/or monocyte population, which comprises a therapeutically effective amount of an antagonist of T-cell immune response cDNA 7 (TIRC7) or of a nucleic acid molecule encoding said antagonist, and optionally a pharmaceutically or cosmetically acceptable carrier.

The present invention also relates to a method of increasing phagocytosis and/or monocyte population, i.e. number, comprising contacting a mammalian cell with an effective amount of T-cell immune response cDNA 7 (TIRC7), an activator of TIRC7 or of a nucleic acid molecule encoding said TIRC7 or said activator, and to a method of decreasing phagocytosis and/or monocyte population, comprising contacting a mammalian cell with an effective amount of an antagonist of T-cell immune response cDNA 7 (TIRC7) or of a nucleic acid molecule encoding said antagonist. These methods can be used for treating therapeutically or prophylacticly a mammal afflicted with a disorder ameliorated by an increase or decrease in phagocytosis and/or monocyte population.

The disorders that can be treated in accordance with the methods of the invention comprise skin disorders, immune system disorders, inflammatory disorders, respiratory disorders, infectious diseases, diabetes, physical wounds, periodontal disorders and central nervous system disorders as well those mentioned in the “background” section.

Furthermore, the present invention relates to an article of manufacture for administering to a mammal the composition of matter of the invention, comprising a solid delivery vehicle having the composition operably affixed thereto.

In addition, the present invention relates to the use of T-cell immune response cDNA 7 (TIRC7) or a fragment thereof, its encoding or regulatory nucleic acid sequences or anti-TIRC7 antibody for targeting monocytes, as a target for diagnosis or therapeutic intervention for diseases related to an increase or decrease in phagocytosis and/or lymphocyte responses, in particular monocyte population in a subject or as a target for screening methods for identifying or isolating agents for the treatment of such diseases.

The present invention also concerns a method of diagnosing any one of the above mentioned disorders comprising:
a) assaying a sample from a subject for TIRC7 transcriptional activity; and
b) determining the existence of the disorder characterized by the induction or suppression of TIRC7 transcriptional activity compared to a healthy subject,
or comprising:
a) assaying a sample from a subject for the presence of TIRC7 protein; and
b) determining the existence of the disorder by the presence of TIRC7 protein, wherein the abnormal presence or absence of TIRC7 protein indicates the presence of the disorder.

The present invention also relates to a method of identifying or isolating a therapeutic agent capable of modulating increase or decrease in phagocytosis and/or monocyte population or increasing lymphocyte response to antigens in a subject comprising a screening method for antagonists/inhibitors or agonist/activators of TIRC7.

In a second aspect, the present invention relates to a method to produce an immunoglobulin or an analog thereof, specific for a desired antigen, which method comprises:
(a) administering said antigen or an immunogenic portion thereof to a nonhuman animal under conditions to stimulate an immune response, whereby said animal produces B cells that secrete immunoglobulin specific for said antigen; wherein said nonhuman animal is characterized by being substantially incapable of producing endogenous T-cell immune response cDNA 7 (TIRC7) or TIRC7 activity; and
(b) recovering said immunoglobulin or analog.

It also relates to the corresponding immortalized B cells, which secrete immunoglobulin binding to a desired antigen, to their cDNAs and to the corresponding nonhuman animals as defined above, preferably for use in antibody production.

In a third aspect, the present invention relates to a vaccine useful for eliciting an immune response to a desired antigen comprising a therapeutically effective amount of an antagonist of T-cell immune response cDNA 7 (TIRC7) or of a nucleic acid molecule encoding said antagonist, optionally further comprising said antigen or immunogenic portion thereof, and optionally further comprising pharmaceutically acceptable agents. In this aspect, said antagonist of TIRC7 is used as an adjuvant.

### DESCRIPTION OF THE FIGURES

FIG. 1. Generation of TIRC7 deficient mice.

(A) Gene targeting in embryonic stem cells. TIRC7 deficient mice were generated by homologous recombination using a vector construct containing the neomycin resistance gene which replaced exons 2-8 of the TIRC7 gene.

(B) PCR analysis of genomic DNA from wild type (+/+), heterozygous (±) and homozygous ((−/−)) mice for the disrupted TIRC7 gene locus. PCR primers were located within the deleted wild type sequence, the neomycin cassette and non-deleted 3′ region. PCR revealed a 1.4 kb fragment (wild type allele) and a 1.2 kb fragment (TIRC7 replaced allele), respectively.

(C) Lack of TIRC7 expression in TIRC7 (−/−) lymphocytes. Flow cytometric analysis in mouse lymphocytes using a cross-reacting human anti-TIRC7 antibody demonstrated significant decrease of TIRC7 expression on heterozygotes (±) and lack of TIRC7 expression on TIRC7 deficient mice ((−/−)) in comparison with wild type littermates (+/+).

(D) TIRC7 deficient mice at day 14 (right) displayed about 30% of the body weight of wild type littermates (left).

(E) Splenocytes isolated from TIRC7 deficient and wildtype (WT) mice were analyzed by flow cytometry to demonstrate lymphocyte subpopulation counts using anti-CD3 FITC, anti-CD4 PerCP, anti-CD8APC, anti-B220 PerCP, anti-CD19 FITC and anti-CD14 conjugated mAbs. Shown is a significant decrease of all resting T and B cell populations as well as monocytes in cells lacking TIRC7 in comparison to WT cells. Although T cell numbers are reduced, no significant changes in CD4/CD8 ratio was observed. For monocyte analysis the gate was set on CD14-positive cells and cell numbers of the WT (188 cells) and TIRC7(−/−) (100 cells) monocyte population are shown in a side scatter vs. CD14 dot plot.

FIG. 2. Histological analysis of TIRC7 (−/−) mice.

(A) Histological staining of TIRC7 (−/−) and WT spleens with hematoxylin and eosin shows a striking hypoplasia of the splenic white pulp of TIRC7(−/−) (KO) spleens compared to WT littermates. Additionally, TIRC7 (−/−) knock out mice show numerous large PALS and small B lymphocytic follicles in comparison to WT mice.

(B) Immunostaining of TIRC7(−/−) and WT spleens revealed a significant hyperplasia of plasma cells within the splenic red pulpa of TIRC7 deficient mice (KO) compared to WT spleens.

FIG. 3. Hyperresponsiveness of T cells from TIRC7 deficient mice.

(A) Proliferation was determined using [³H] thymidine in splenocytes isolated from TIRC7 knock out (KO) and wild type (WT) mice. Cells were activated for 48 h with either anti-CD3 mAb alone or in combination with anti-CD28 mAb (a) or PHA (b) at different concentrations. Unstimulated T cells of wild type (WTo) and TIRC7 deficient mice (KOo) served as controls. Compared to wild type cells TIRC7 deficient cells exhibited hyperreactivity in response to activation stimuli (KO st).

(B) Splenocytes were isolated and either remained non-stimulated (WTo, KOo) or were activated (WTs; KO st) with PHA for 48 h, and culture supernatants collected after 48 h. IL-2 and IFN-γ cytokine production was determined by ELISA in the supernatants of splenocytes from wild type and TIRC7 (−/−) mice. The results shown are representative of three different wild type and TIRC7 deficient animals, respectively. Significantly elevated cytokine levels are observed in all TIRC7 deficient cells.

FIG. 4. Analysis of the Expression Profile of Activation Markers on resting T cells isolated from TIRC7 deficient and Wild-Type Mice.

(A) The expression of CD69 and CD25 as well as CD62L and CD44 was determined in T lymphocytes isolated from wild type (+/+) and TIRC7 deficient ((−/−)) mice by flow cytometry analysis with FITC labeled anti-CD3 mAb and PE labeled antibody, respectively. The percentages of cells positive for the respective marker molecule are indicated in the right upper quadrant of each plot. The gate was set on CD4-positive cells using anti-CD4-PerCP mAb.

(B) Determination of CD11a expression was performed by FACS on resting and activated T lymphocytes, isolated from TIRC7 knock out (−/−) and WT (+/+) mice using FITC-conjugated anti-CD3 mAb and PE-conjugated CD11a mAb. Percentages of the naive and memory cell populations are indicated by boxes in the upper right quadrant of each plot.

(C) The CTLA-4 expression in splenocytes isolated from TIRC7(−/−) and wild type mice was analyzed by FACS. T cells were stained using FITC labeled anti-CD3 mAb combined with PE labeled mouse anti-CTLA-4 mAb. The gate was set on CD4-positive cells using anti-CD4-PerCP mAb. Shown is the CTLA-4 expression on the surface of unstimulated cells (a) which is only minimally increased in TIRC7 deficient mice (−/−) (b) upon 48 h PHA activation compared with wild type littermates (+/+). Also, insufficient intracellular CTLA-4 expression is observed in TIRC7 deficient mice compared to wild type littermates (c).

(D) CD28 expression was determined by staining splenocytes isolated from wild type (+/+) and TIRC7 deficient (−/−) mice with anti-CD3-FITC labeled mAb and anti-CD28-PE labeled 20 mAb. ICOS staining was performed using ICOS Ab followed by staining with secondary goat-anti-mouse-PE labelled Ab.

(E) The expression of CD71 was determined in splenocytes isolated from wild type (+/+) and TIRC7 deficient (−/−) mice by flow cytometry analysis gated on CD4 and stained with anti-CD3-FITC labeled mAb and anti-CD71-PE labeled mAb 48 h after activation with PHA. The percentage of cells positive for CD71 is indicated in the right upper quadrant of each plot.

FIG. 5. In Vivo T cell response to antigen of TIRC7-Deficient Mice.

(A) Delayed-type hypersensitivity (DTH) response to antigen (ovalbumin) was estimated by measuring foot pad thickness of WT and TIRC7(−/−) mice 48 h after re-challenge with ovalbumin. The percentage differences in the swellings of the foot pads between ova- and PBS-injected control animals were estimated. TIRC7(−/−) mice showed a significantly swelling between the right and left foot pads compared to WT.

(B) Histology of foot pad-skin obtained from wild type mice (WT) shows expected mild parenchymal lymphocyte infiltration in dermis (d) whereas TIRC7(−/−) mice (KO) show a severe perivascular and parenchymal infiltration in stratum reticulare. The sections were isolated after 48 h second antigen challenge of DTH response and stained with hematoxylin and eosin ((e) epidermis). Shown is 100× magnification.

FIG. 6. Increased B cell activation in TIRC7 (−/−) mice.

(A) Proliferation of B cells following incubation with various stimuli, i.e. with anti-CD40 antibody alone and with LPS in combination with IL-4, exhibited much higher levels of TIRC7(−/−) B cell (KO st) response compared to those of WT littermates (WTst) by Thymidine incorporation assay, respectively. (WTo and KOo represent non stimulated populations as controls).

(B) Immunoglobulin concentrations in culture supernatants of anti-CD40 stimulated splenocytes isolated from WT and TIRC7 deficient mice show significant higher levels of IgM and IgG secreted by TIRC7(−/−) B cells (KO st) compared to stimulated WT (WTst) or nonstimulated controls (WTo, KOo). Ig concentrations in the supernatants were determined by ELISA after 7 days of stimulation.

(C) The activation status of B cells was examined by determining the levels of various IgGs in the serum of wild type and TIRC (−/−) mice (▴) using ELISA. The results from three different animals, wild type (A) and TIRC7 deficient, respectively, show elevated levels of all immunoglobulins examined in TIRC7(−/−) mice.

(D) Expression of costimulatory molecule CD86 on B cells after 24 h LPS/IL-4 in vitro stimulation was analyzed by FACS. Staining with anti-B220 PerCP, and anti-CD86 PE conjugated mAb shows that CD86 expression is already upregulated in resting status on the surface of cells from TIRC7 (−/−) deficient mice. Arrows indicate the percentages of the CD86-high population on activated B cells in the boxes.

FIG. 7. Macrophages revealed extensive morphological and functional defects in TIRC7 deficient mice

(A) Macrophages isolated from the peritoneal cavities of TIRC7(−/−) and wild type mice remained non stimulated or were stimulated with LPS and IL-4 for 48 h. TIRC7 deficient mice revealed significant lower numbers of macrophages as can be seen in the unstimulated cell populations. After stimulation, TIRC7(−/−) macrophages showed different morphology of proliferating macrophages compared to WT littermates.

(B) Confocal microscope images illustrating immunostaining for cytoskeleton proteins such as tubulin (a), vinculin (b) and alpha-actin (c) in macrophages obtained from TIRC7(−/−) and wild type mice (+/+) demonstrated decreased expression of all these proteins in TIRC7 deficient cells (−/−).

### DETAILED DESCRIPTION OF THE INVENTION

This invention is based on the discovery that TIRC7 deficient mice exhibit increased T and B cell proliferative response to different stimuli in vitro and in vivo compared to wild type littermates. The expression of T cell surface molecules such as CD69 and CD25 demonstrated only a moderate increase whereas CD62L and CD44 were found to be slightly decreased and elevated, respectively, in TIRC7 deficient cells compared to wild type. Strikingly, the expression of costimulatory molecules such as CTLA4, CD28 and ICOS was significantly reduced whereas no significant changes in expression kinetics was observed for PD1 and CD40L in TIRC7 deficient T cells compared to their littermates. B cell proliferation as well as immunoglobulin expression were induced in TIRC7 (−/−) mice splenocytes following activation with IL-4 and LPS. Expression of CD86 was increased in TIRC7 deficient resting B cells whereas CD80 and CD40 expression remained unchanged. The monocyte fraction exhibited a decrease in numbers and failure of phagocytosis and abnormal cytoskeleton architecture. These results demonstrate that TIRC7 function is essential for regulating the immune response to various antigens.

This ability to specifically increase and decrease these cellular functions by modulating TIRC7 expression and/or activity permits the treatment and prevention of disorders, which would be ameliorated by an increase, or decrease of phagocytosis and/or monocytes. Accordingly, this invention relates a composition of matter for treating therapeutically or prophylacticly a mammal afflicted with a disorder ameliorated by an increase in phagocytosis and/or monocyte population, which comprises a therapeutically effective amount of T-cell immune response cDNA 7 (TIRC7), an activator of TIRC7 or of a nucleic acid molecule encoding said TIRC7 or said activator, and optionally a pharmaceutically or cosmetically acceptable carrier.

Conclusively, the present invention also relates to a composition of matter for treating therapeutically or prophylacticly a mammal afflicted with a disorder ameliorated by a decrease in phagocytosis and/or monocyte population, which comprises a therapeutically effective amount of an antagonist of T-cell immune response cDNA 7 (TIRC7) or of a nucleic acid molecule encoding said antagonist, and optionally a pharmaceutically or cosmetically acceptable carrier.

The term “TIRC7” as used in accordance with the present invention, denotes a protein which initially has been described to be involved in the signal transduction of T-cell activation and proliferation and that, preferably in a soluble form is capable of inhibiting or suppressing T-cell proliferation in response to alloactivation in a mixed lymphocyte culture or in response to mitogens when exogeneously added to the culture. In vitro translated TIRC7 protein has been shown to be able to efficiently suppress the proliferation of T-cells in a dose dependent manner in response to alloactivation in a mixed lymphocyte culture or in response to mitogens. TIRC7 is known to the person skilled in the art and described, inter alia, in WO99/11782, Utku, Immunity 9 (1998), 509-518 and Heinemann, Genomics 57 (1999), 398-406, which also disclose the amino and nucleic acid sequences of TIRC7.

The agent in the instant compositions that specifically increases or decreases TIRC7 expression and/or activity can be any type of compound known in the art. Examples include, without limitation, organic molecules, inorganic molecules, peptides, proteins, carbohydrates, nucleic acid molecules, lipids, and any combination thereof. TIRC7 antisense nucleic molecules, for example, can be used to decrease phagocytosis. TIRC7 expression vectors or TIRC7 ligands, for example, can be used to increase phagocytosis or monocyte population.

Techniques that can be used for increasing or decreasing the phagocytic activity of cells in a mammal by modulating TIRC7 activity in accordance with the present invention can be derived from the prior art. For example, in WO95/09011 alternatively to the present invention it is proposed to introduce into appropriate cells a DNA molecule coding for an Fc receptor so that said DNA molecule is expressed and said Fc receptor thereby produced and the phagocytic activity of said cells thereby increased. Similarly, but in accordance with the present invention TIRC7 encoding DNA would be used. Other approaches that may be modified and used in accordance with present invention are described for example in WO96/40199 and WO95/09002.

As used herein, the term “mammal” means any member of the higher vertebrate animals included in the class Mammalia, as defined in Webster's Medical Desk Dictionary 407 (1986), and includes but is not limited to humans, other primates, pigs, dogs, and rodents (such as immune suppressed mice). In the preferred embodiment of this invention, the mammal is a human.

The instant composition of matter can be of any form known in the art. In one embodiment, the composition comprises a pharmaceutically acceptable carrier and one or more discrete pharmaceutical compounds that function as the agent that specifically alters TIRC7 expression and/or activity. In another embodiment, the composition of matter comprises a naturally-occurring composition, or an extract or component thereof, which is deemed pharmaceutically or cosmetically acceptable. Such naturally occurring compositions contain certain components which function as active agents, and numerous others that serve as pharmaceutical or cosmetically carriers. The instant compositions can be artificial, naturally occurring, or a combination thereof. In addition, the compositions can be of any physical form known in the art, such as liquids (e. g., solutions, creams, lotions, gels, injectables), solids (e. g., tablets, capsules, powders, granules), aerosols, and coatings.

The terms “antagonist/inhibitor and agonist/activator” in accordance with the present invention include chemical agents that modulate the action of TIRC7, either through altering its enzymatic or biological activity or through modulation of expression, e.g., by affecting transcription or translation. In some cases the antagonist/inhibitor or agonist/activator may also be a substrate or ligand binding molecule.

The term “activator,” as used herein, includes both substances necessary for TIRC7 to become active in the first place, and substances which merely accentuate its activity.

The term “inhibitor” includes both substances which reduce the activity of the TIRC7 and those which nullify it altogether. When more than one possible activity is defined herein for TIRC7, the inhibitor or activator may modulate any or all of TIRC7 activities. An “antagonist” or “agonist” that modulates the activity of TIRC7 and causes for example a response in a cell based assay refers to a compound that alters directly or indirectly the activity of TIRC7 or the amount of active TIRC7. Typically, the effect of an antagonist is substantially the same as that of the anti-TIRC7 antibodies described in Utku, Immunity 9 (1998), 509-518. Antagonists include competitive as well as non-competitive antagonists. A competitive antagonist (or competitive blocker) interacts with or near the site specific for agonist binding. A non-competitive antagonist or blocker inactivates the function of the receptor by interacting with a site other than the agonist interaction site. Preferably, the antagonist/inhibitor and agonist/activator of TIRC7 are small chemical agents which directly interact with TIRC7. Therefore, there will preferably be a direct relationship between the molar amount of compound required to inhibit or stimulate TIRC7 activity and the molar amount of TIRC7 present or lacking in the cell.

Activators and inhibitors may be designed by structure-assisted computer modeling for example based on alpha-helix and alpha-helix forming regions (“alpha-regions”), beta-sheets and beta-sheet-forming regions (“beta-regions”), turns and turn-forming regions (“turn-regions”), coils and coil-forming regions (“coil-regions”), hydrophilic regions, hydrophobic regions, alpha amphipathic regions, beta amphipathic regions, flexible regions, surface-forming regions, substrate binding region, and high antigenic index regions. Such preferred regions include Garnier-Robson alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha and beta amphipathic regions, Karplus-Schulz flexible regions, Emini surface-forming regions, and Jameson-Wolf high antigenic index regions. Computer predictions can be made made using for example GCG-software derived from HGMP resource center Cambridge (Rice, 1995)

Said agonist/activator of TIRC7 can be or can be derived from, for example, a TIRC7 polypeptide, a TIRC7 gene, an anti-TIRC7 antibody, a transcription regulator of the TIRC7 gene or a ligand binding molecule, a TIRC7 ligand, or a cell (over)expressing TIRC7. Preferably, said TIRC7 polypeptide is a recombinant TIRC7, a functional derivative thereof or a functionally equivalent substance. DNA sequences encoding TIRC7 as well as functional derivatives and functionally equivalent substances which can be used in the methods and uses of the invention are described in the prior art; see the references cited above. Moreover, DNA and amino acid sequences of TIRC7 are available in the Gene Bank database. As described above, methods for the production of recombinant proteins are well-known to the person skilled in the art; see, e.g., Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1989), (1994).

TIRC7 antagonists may be peptides, proteins, nucleic acids, a TIRC7 gene targeting vector, antibodies, small organic compounds, peptide mimics, aptamers or PNAs (Milner, Nature Medicine 1 (1995), 879-880; Hupp, Cell 83 (1995), 237-245; Gibbs, Cell 79 (1994), 193-198; Gold, Ann. Rev. Biochem. 64 (1995), 736-797). For the preparation and application of such compounds, the person skilled in the art can use the methods known in the art, for example those referred to above. Furthermore, antagonists/inhibitors of TIRC7 and methods for obtaining the same are described in, for example, PCT/EP01/12485.

Nucleic acid molecules specifically hybridizing to TIRC7 encoding genes and/or their regulatory sequences may be used for repression of expression of said gene, for example due to an antisense or triple helix effect or they may be used for the construction of appropriate ribozymes (see, e.g., EP-B1 0 291 533, EP-A1 0 321 201, EP-A2 0 360 257) which specifically cleave the (pre)-mRNA of a gene encoding TIRC7. The nucleic acid sequence encoding TIRC7 is known in the art; see references supra. Selection of appropriate target sites and corresponding ribozymes can be done as described for example in Steinecke, Ribozymes, Methods in Cell Biology 50, Galbraith et al. eds Academic Press, Inc. (1995), 449-460. Furthermore, methods are described in the literature for identifying nucleic acid molecules such as an RNA fragment that mimics the structure of a defined or undefined target RNA molecule to which a compound binds inside of a cell resulting in retardation of cell growth or cell death; see, e.g., WO 98/18947 and references cited therein. These nucleic acid molecules can be used to identify unknown compounds of pharmaceutical interest, and to identify unknown RNA targets for use in treating a disease. Alternatively, for example, the conformational structure of the RNA fragment which mimics the binding site can be employed in rational drug design to modify known ligands to make them bind more avidly to the target. One such methodology is nuclear magnetic resonance (NMR), which is useful to identify drug and RNA conformational structures. Still other methods are, for example, the drug design methods as described in WO 95/35367, U.S. Pat. No. 5,322,933, where the crystal structure of the RNA fragment can be deduced and computer programs are utilized to design novel binding compounds which can act as antibiotics.

Nucleic acid sequences that are complementary to the TIRC7 encoding gene sequence or sense nucleic acid sequences can be synthesized for antisense therapy. These sense or antisense molecules may be DNA, stable derivatives of DNA such as phosphorothioates or methylphosphonates, RNA, stable derivatives of RNA such as 2′-O-alkylRNA, or other TIRC7 antisense oligonucleotide mimetics. TIRC7 antisense molecules may be introduced into cells by microinjection, liposome encapsulation or by expression from vectors harboring the antisense sequence. TIRC7 antisense therapy may be particularly useful for the treatment of diseases where it is beneficial to reduce TIRC7 activity. TIRC7 gene therapy may be used to introduce TIRC7 into the cells of target organisms. The TIRC7 gene can be ligated into viral vectors that mediate transfer of the TIRC7 DNA by infection of recipient host cells. Suitable viral vectors include retrovirus, adenovirus, adeno-associated virus, herpes virus, vaccinia virus, polio virus and the like. Alternatively, TIRC7 DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo as well as in vivo TIRC7 gene therapy. TIRC7 gene therapy may be particularly useful for the treatment of diseases where it is beneficial to elevate TIRC7 activity. Protocols for molecular methodology of gene therapy applicable to the TIRC7 gene are described in Gene Therapy Protocols, edited by Paul D. Robbins, Human press, Totawa N.J., 1996.

Furthermore, the so-called “peptide nucleic acid” (PNA) technique can be used for the inhibition of the expression of a gene encoding a TIRC7. For example, the binding of PNAs to complementary as well as various single stranded RNA and DNA nucleic acid molecules can be systematically investigated using, e.g., thermal denaturation and BIAcore surface-interaction techniques (Jensen, Biochemistry 36 (1997), 5072-5077). The synthesis of PNAs can be performed according to methods known in the art, for example, as described in Koch, J. Pept. Res. 49 (1997), 80-88; Finn, Nucleic Acids Research 24 (1996), 3357-3363. Furthermore, folding simulations and computer redesign of structural motifs of TIRC7 and its receptors or ligands can be performed as described above to design drugs capable of inhibiting the biological activity of TIRC7.

Preferably, antibodies can be employed in accordance with the present invention specifically recognizing TIRC7, or antibody receptors or parts, i.e. specific fragments or epitopes, of such TIRC7s and ligands thereby inactivating the TIRC7 or the TIRC7 ligand. These antibodies can be monoclonal antibodies, polyclonal antibodies or synthetic antibodies as well as fragments of antibodies, such as Fab, Fv or scFv fragments etc. Antibodies or fragments thereof can be obtained by using methods which are described, e.g., in Harlow and Lane “Antibodies, A Laboratory Manual”, CSH Press, Cold Spring Harbor, 1988 or EP-B1 0 451 216 and references cited therein. For example, surface plasmon resonance as employed in the BIAcore system can be used to increase the binding efficiency of phage antibodies which bind to an epitope of the TIRC7 or its ligand (Schier, Human Antibodies Hybridomas 7 (1996), 97-105; Malmborg, J. Immunol. Methods 183 (1995), 7-13).

Putative inhibitors which can be used in accordance with the present invention including peptides, proteins, nucleic acids, antibodies, small organic compounds, ligands, hormones, peptide mimetics, PNAs and the like capable of inhibiting the biological activity of TIRC7 or its ligand may be identified according to the methods known in the art, for example as described in EP-A-0 403 506.

In a preferred embodiment of the present invention, the antagonist is a nucleic acid molecule and designed to be expressed in monocytes.

In a further preferred embodiment, the antagonist blocks an interaction of TIRC7 and its ligand. Preferably, said antagonist is or comprises

(i) an anti-TIRC7 antibody or an anti-TIRC7-ligand antibody; or

(ii) a non-stimulatory form of TIRC7 or of its ligand.

An anti-TIRC7 antibody to be used in accordance with pharmaceutical compositions of the present invention can be preferably a monoclonal antibody, but also include a polyclonal antibody, a single chain antibody, human or humanized antibody, primatized, chimerized or a fragment thereof that specifically binds TIRC7 peptide or polypeptide also including bispecific antibody, synthetic antibody, antibody fragment, such as Fab, Fv or scFv fragments etc., or a chemically modified derivative of any of these. The general methodology for producing antibodies is well-known and has been described in, for example, Köhler and Milstein, Nature 256 (1975), 494 and reviewed in J. G. R. Hurrel, ed., “Monoclonal Hybridoma Antibodies: Techniques and Applications”, CRC Press Inc., Boco Raron, Fla. (1982), as well as that taught by L. T. Mimms et al., Virology 176 (1990), 604-619; see also infra.

Further sources for the basic structure of inhibitors can be employed and comprise, for example, mimetic analogs of the TIRC7 polypeptide. Mimetic analogs of the TIRC7 polypeptide can be generated by, for example, substituting the amino acids that are expected to be essential for the biological activity with, e.g., stereoisomers, i.e. D-amino acids; see e.g., Tsukida, J. Med. Chem. 40 (1997), 3534-3541. Furthermore, the TIRC7 polypeptide can be used to identify synthetic chemical peptide mimetics that bind to or can function as a ligand, substrate, binding partner or the receptor of the TIRC7 polypeptide as effectively as does the natural polypeptide; see, e.g., Engleman, J. Clin. Invest. 99 (1997), 2284-2292. For example, folding simulations and computer redesign of structural motifs of the protein of the invention can be performed using appropriate computer programs (Olszewski, Proteins 25 (1996), 286-299; Hoffman, Comput. Appl. Biosci. 11 (1995), 675-679). Computer modelling of protein folding can be used for the conformational and energetic analysis of detailed peptide and protein models (Monge, J. Mol. Biol. 247 (1995), 995-1012; Renouf, Adv. Exp. Med. Biol. 376 (1995), 37-45). In particular, the appropriate programs can be used for the identification of interactive sites of the TIRC7 polypeptide and its ligand or other interacting proteins by computer assistant searches for complementary peptide sequences (Fassina, Immunomethods (1994), 114-120. Further appropriate computer systems for the design of protein and peptides are described in the prior art, for example in Berry, Biochem. Soc. Trans. 22 (1994), 1033-1036; Wodak, Ann. N. Y. Acad. Sci. 501 (1987), 1-13; Pabo, Biochemistry 25 (1986), 5987-5991. Methods for the generation and use of peptide mimetic combinatorial libraries are described in the prior art, for example in Ostresh, Methods in Enzymology 267 (1996), 220-234 and Dorner, Bioorg. Med. Chem. 4 (1996), 709-715. Furthermore, a three-dimensional and/or crystallographic structure of the TIRC7 protein can be used for the design of mimetic inhibitors of the biological activity of the protein of the invention (Rose, Biochemistry 35 (1996), 12933-12944; Rutenber, Bioorg. Med. Chem. 4 (1996), 1545-1558).

It is also well known to the person skilled in the art, that it is possible to design, synthesize and evaluate mimetics of small organic compounds that, for example, can act as a substrate or ligand to the TIRC7 polypeptide. For example, it has been described that D-glucose mimetics of hapalosin exhibited similar efficiency as hapalosin in antagonizing multidrug resistance assistance-associated protein in cytotoxicity; see Dinh, J. Med. Chem. 41 (1998), 981-987.

Recombinant TIRC7 polynucleotides, antisense molecules and vectors incorporating such polynucleotides or antisense molecules can be produced by methods known to those skilled in molecular biology. For example, the choice of vectors which would depend on the function desired and include plasmids, cosmids, viruses, bacteriophages and other vectors used conventionally in genetic engineering. Methods which are well known to those skilled in the art can be used to construct various plasmids and vectors; see, for example, the techniques described in Sambrook, and Ausubel cited supra. Alternatively, the polynucleotides and vectors can be reconstituted into liposomes for delivery to target cells. Relevant sequences can be transferred into expression vectors where expression of a particular polypeptide is required. Typical cloning vectors include pBscpt sk, pGEM, pUC9, pBR322 and pGBT9. Typical expression vectors include pTRE, pCAL-n-EK, pESP-1, pOP13CAT, pET, pGEX, pMALC, pPIC9, pBac.

The antibodies, nucleic acid molecules, inhibitors and activators used in the compositions of the present invention preferably have a specificity at least substantially identical to the binding specificity of the natural ligand or binding partner of the TIRC7 protein, in particular if TIRC7 stimulation is desired. An antibody or inhibitor can have a binding affinity to the TIRC7 protein of at least 10⁵ M⁻¹, preferably higher than 10⁷ M⁻¹ and advantageously up to 10¹⁰ M⁻¹ in case TIRC7 suppression should be mediated.

In a preferred embodiment, a suppressive antibody or inhibitor has an affinity of at least about 10⁻⁷ M, preferably at least about 10⁻⁹ M and most preferably at least about 10⁻¹ M; and a TIRC7 stimulating activator has an affinity of less than about 10⁻⁷ M, preferably less than about 10⁻⁶ M and most preferably in order of 10⁻⁵M.

Disorders that can be treated or prevented using the instant invention include any disorder that can be ameliorated (i.e., a positive effect on the disorder per se, and/or its secondary effects) by either an increase or decrease in phagocytosis or monocyte population. These disorders include, without limitation, immune system disorders, diabetes, inflammatory disorders, disorders of the central nervous system, skin disorders, physical wounds, periodontal disorders and respiratory disorders. A number of disorders have characteristics of more than one category of disorder. Such disorders include, for example, adhesion disorders, which can be categorized as both skin disorders and immune system disorders. Accordingly, a statement herein that a disorder is of a particular category (e.g., skin disorder) means that, at the very least, the disorder bears traits of that category. Again, however, the disorder may additionally bear traits of another category. Increasing the ability of immune cells to ingest foreign objects like bacteria and viruses would be expected to enhance the immune response. For example, mononuclear phagocytes are inactive in chronic microbial infections (Reiner, Immunol. Today 15 (1994), 37481), and their re-activation would be expected to treat the disease. Alternatively, disorders wherein the immune system is too active would be ameliorated by inhibiting phagocytosis.

Immune system and inflammatory disorders treatable in this invention include, by way of example, AIDS, chemotherapy-induced immunodeficiency, asthma, damage due to toxic substance exposure (e.g., asbestos or smoke), host rejection of implants and transplanted tissue, adhesion disorders, mild infections (such as common colds), severe infections (such as meningitis or “killer bacteria”), wounds (such as infected, diabetic, acute and chronic wounds), restenosis, cystic fibrosis, pulmonary emphysema, periodontal disease, and diaper rash. Skin disorders include unwanted pigmentation, unwanted de-pigmentation, psoriasis, rashes, and certain physical skin imperfections (e.g., wrinkles). In one specific example, vitiligo patients are treated with melanin (via liposomes or plain) together with a phagocytosis-increasing agent to darken the light spots. Alternatively, they are treated with an agonist of TIRC7 to lighten the darker sites. In an example related to skin disorders, gray hair is treated with melanin (plain or liposome-delivered) and a phagocytosis increasing agent, ideally in a shampoo or cream. Central nervous system disorders include, without limitation, Alzheimer's disease and other senile plaque disorders (treated via up-regulating the phagocytosis of amyloid fibrils), depression, phobic disorders, and other disorders resulting from secondary effects of benzodiazepine treatment.

Hence, the present invention provides a method of increasing phagocytosis and/or monocyte population, comprising contacting a mammalian cell with an effective amount of T-cell immune response cDNA 7 (TIRC7), an activator of TIRC7 or of a nucleic acid molecule encoding said TIRC7 or said activator. This method may comprise
(a) obtaining cells, tissue or an organ from a subject;
(b) introducing into said cells, tissue or organ a nucleic acid molecule encoding and capable of expressing TIRC7 or its ligand in vivo; and
(c) reintroducing the cells, tissue or organ obtained in step (b) into the same subject or a different subject.

It is envisaged by the present invention that TIRC7 and the nucleic acid molecules encoding TIRC7 or entities of the corresponding activator are administered either alone or in combination, and optionally together with a pharmaceutically acceptable carrier or exipient. Said nucleic acid molecules may be stably integrated into the genome of the cell or may be maintained in a form extrachromosomally, see, e.g., Calos, Trends Genet. 12 (1996), 463-466. On the other hand, viral vectors described in the prior art may be used for transfecting certain cells, tissues or organs. Furthermore, it is possible to use a pharmaceutical composition of the invention which comprises a nucleic acid molecule encoding a TIRC7 in gene therapy. Suitable gene delivery systems may include liposomes, receptor-mediated delivery systems, naked DNA, and viral vectors such as herpes viruses, retroviruses, adenoviruses, and adeno-associated viruses, among others. Delivery of nucleic acid molecules to a specific site in the body for gene therapy may also be accomplished using a biolistic delivery system, such as that described by Williams (Proc. Natl. Acad. Sci. USA 88 (1991), 2726-2729).

Standard methods for transfecting cells with nucleic acid molecules are well known to those skilled in the art of molecular biology, see, e.g., WO94/29469. Gene therapy to prevent or decrease the development of diseases described herein may be carried out by directly administering the nucleic acid molecule encoding TIRC7 to a patient or by transfecting cells with said nucleic acid molecule ex vivo and infusing the transfected cells into the patient.

Furthermore, research pertaining to gene transfer into cells of the germ line is one of the fastest growing fields in reproductive biology. Gene therapy, which is based on introducing therapeutic genes into cells by ex-vivo or in-vivo techniques is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO94/29469; WO97/00957 or Schaper, Current Opinion in Biotechnology 7 (1996), 635-640, and references cited therein. The nucleic acid molecules comprised in the pharmaceutical composition of the invention may be designed for direct introduction or for introduction via liposomes, or viral vectors (e.g. adenoviral, retroviral) containing said nucleic acid molecule into the cell. Preferably, said cell is a germ line cell, embryonic cell, or egg cell or derived therefrom.

Thus, in a preferred embodiment, the nucleic acid molecule comprised in the pharmaceutical composition for the use of the invention is designed for the expression of TIRC7 by cells in vivo by, for example, direct introduction of said nucleic acid molecule or introduction of a corresponding plasmid, a plasmid in liposomes, or a viral vector (e.g. adenoviral, retroviral) containing said nucleic acid molecule.

Furthermore, the present invention provides a method to decrease phagocytosis and/or monocyte population, comprising contacting a mammalian cell with an effective amount of an antagonist of T-cell immune response cDNA 7 (TIRC7) or of a nucleic acid molecule encoding said antagonist; see supra.

The present invention further provides methods of treatment and prophylaxis regarding mammals affected by a disorder ameliorated by an increase in phagocytosis and/or monocyte population, which comprises administering to the mammal a therapeutically effective amount of T-cell immune response cDNA 7 (TIRC7), an activator of TIRC7 or of a nucleic acid molecule encoding said TIRC7 or said activator; see supra.

In addition, the present invention provides a method of treating or preventing in a mammal afflicted with a disorder ameliorated by a decrease in phagocytosis and/or monocyte population, which comprises administering to the mammal a therapeutically effective amount of an antagonist of T-cell immune response cDNA 7 (TIRC7) or of a nucleic acid molecule encoding said antagonist.

Said antagonist or activator for use in the mentioned methods can be any agent as described above.

The mammalian cells treated in the instant methods are preferably TIRC7 expressing cells, and include, without limitation, keratinocytes, fibroblasts, and “professional phagocytes” (i.e., cells having phagocytosis as a primary function). Professional phagocytes include, for example, neutrophils, macrophages and macrophage-like cells (e.g., Langerhans cells and Kupfer cells). In the preferred embodiment, the mammalian cells are human cells.

In this invention, the “appropriate cells” in which phagocytosis and TIRC7 expression have to be altered in response to the instant compositions of matter are readily determined based on the nature of the disorder being treated or prevented. For example, if the disorder being treated is a pigmentation disorder, the appropriate cells in which TIRC7 expression or activity needs to be altered are keratinocytes.

The instant methods are directed at preventing as well as treating disorders. As used herein, “therapeutically treating” a disorder means reducing the disorder's progression, ceasing the disorder's progression, ceasing or otherwise ameliorating secondary effects of the disorder, reversing the disorder's progression, or preferably, curing the disorder. As used herein, “prophylactly treating” a disorder means reducing, and preferably eliminating, the likelihood of the disorder's occurrence or of occurrence of secondary effects.

In this invention, administering the instant compositions can be alcohols and amino acids, hydrophilic polymers (e.g., polycarbophil and polyvinylpyrolidone), and adhesives and tackifiers (e.g., polyisobutylenes, silicone-based adhesives, acrylates and polybutene). Topical delivery of some of the compositions of this invention, particularly those comprising proteins or nucleic acid molecules such antisense nucleic molecules or TIRC7 expression vectors, can be achieved using liposomes. The liposomes are preferably nonionic. In one example, they contain (a) glycerol dilaurate; (b) compounds having the steroid backbone found in cholesterol; and (c) fatty acid ethers having from about 12 to about 18 carbon atoms, wherein the constituent compounds of the liposomes are in a ratio of about 37.5:12.5:33.3:16.7. Liposomes comprising glycerol dilaurate/cholesterol/polyoxyethylene-10stearyl ether/polyoxyethylene-9-lauryl ether (“GDL” liposomes) are preferred. In one embodiment, the liposomes are present in an amount, based upon the total volume of the composition, of from about 10 mg/ml to about 100 mg/ml, and preferably from about 15 mg/ml to about 50 mg/ml. A ratio of about 37.5:12.5:33.3:16.7 is preferred. Methods of preparing liposomes are well known in the art, such as those disclosed in Niemiec, Pharm. Res. 12 (1995), 1184-1188. Also, for topical or transdermal administration, the instant compositions can be combined with other components such as moisturizers, cosmetic adjuvants, anti-oxidants, bleaching agents, tyrosinase inhibitors and other known depigmentation agents, alpha-hydroxy acids, surfactants, foaming agents, conditioners, humectants, fragrances, viscosifiers, buffering agents, preservatives, sunscreens and the like. The compositions of this invention can also contain active amounts of retinoids including, for example, tretinoin, retinol, esters of tretinoin and/or retinol and the like.

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Systems for central nervous system delivery include, for example, a lipidcoupled derivative to cross the blood brain barrier (e.g. DHA). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Such delivery systems also include, for example, toothpaste, mouthwash, lozenges and lollipops.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, ascorbic acid, and natural extracts), anti-caking agents, coating agents, and chelating agents (e.g., EDTA). Oil-in-water emulsions, water-in-oil emulsions, solvent-based formulations and aqueous gels known to those of skill in the art can also be utilized as vehicles for the delivery of the compositions of this invention.

This invention still further provides an article of manufacture for administering to a mammal the instant composition of matter, comprising a solid delivery vehicle having the composition operably (i.e., deliverably) affixed thereto. The solid delivery vehicle can be any device designed to come into temporary or permanent contact with the body, whether or not it was originally intended for use as a delivery vehicle. Examples of the instant article of manufacture include, without limitation, coated bandages or other wound dressing for treating wounds, coated bodily implants (including implants with coated internal scaffolding) for either preventing or promoting tissue growth, and coated balloon catheters and stents for preventing restenosis.

In addition, this invention provides a method of administering a therapeutic; prophylactic or cosmetic compound to a mammal, comprising administering to the mammal (a) the compound and (b) a composition of matter of the invention comprising a pharmaceutical or cosmetic carrier and an agent that specifically modulates TIRC7 expression and/or activity in an amount sufficient to increase phagocytosis in cells where uptake of the compound is desired, wherein the composition is administered prior to and/or concurrently with the administration of the compound. The pharmaceutical compound can be, for example, a polypeptide, protein, or nucleic acid molecule. In one embodiment, the pharmaceutical compound and composition are administered together via microscopic porous biodegradable beads, which then release the pharmaceutical compound after being ingested through phagocytosis by the appropriate cells.

In accordance with the above, the present invention also relates to the use of T-cell immune response cDNA 7 (TIRC7) or a fragment thereof, its encoding or regulatory nucleic acid sequences or anti-TIRC7 antibody for targeting monocytes, as a target for diagnosis or. therapeutic intervention for diseases related to an increase or decrease in phagocytosis and/or monocyte population in a subject or as a target for screening methods for identifying or isolating agents for the treatment of such diseases.

Pharmaceutically useful compositions such as described herein-before, comprising TIRC7 DNA, TIRC7 RNA, or TIRC7 protein, or modulators of TIRC7 activity, i.e. activator/agonist or inhibitor/antagonist, or chemical derivatives thereof may be formulated according to known methods such as by the admixture of a pharmaceutically acceptable carrier. Examples of such carriers and methods of formulation may be found in Remington's Pharmaceutical Sciences. To form a pharmaceutically acceptable composition suitable for effective administration, such compositions will contain an effective amount of the protein, DNA, RNA, or modulator. Therapeutic or diagnostic compositions of the invention are administered to an individual in amounts sufficient to treat or diagnose disorders in which modulation of TIRC7-related activity is indicated. The effective amount may vary according to a variety of factors such as the individual's condition, weight, sex and age. Other factors include the mode of administration. The pharmaceutical compositions may be provided to the individual by a variety of routes such as by intracoronary, intraperitoneal, subcutaneous, intravenous, transdermal, intrasynovial, intramuscular or oral routes.

The term “chemical derivative” describes a molecule that contains additional chemical moieties that are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

TIRC7 DNA, TIRC7 RNA, or TIRC7 protein, or modulators of TIRC7 activity disclosed herein may be used alone at appropriate dosages defined by routine testing in order to obtain optimal activation or inhibition of the TIRC7 activity while minimizing any potential toxicity. In addition, co-administration or sequential administration of other agents may be desirable.

A therapeutically effective dose refers to that amount of protein, antibodies, nucleic acid, agonists, activators, antagonists, or inhibitors which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio between therapeutic and toxic effects is the therapeutic index, and it can be expressed as the ratio, LD50/ED50.

In a further embodiment the present invention relates to a method of diagnosing a disorder related to an increase or decrease in phagocytosis and/or monocyte population in a subject comprising:
a) assaying a sample from a subject for TIRC7 transcriptional activity; and
b) determining the existence of the disorder characterized by the induction or suppression of TIRC7 transcriptional activity compared to a healthy subject.

In a still further embodiment the present invention relates to a method of diagnosing a disorder related to an increase or decrease in phagocytosis and/or monocyte population in a subject comprising:
a) assaying a sample from a subject for the presence of TIRC7 protein; and
b) determining the existence of the disorder by the presence of TIRC7 protein, wherein the abnormal presence or absence of TIRC7 protein indicates the presence of the disorder.

Preferably, in said methods the cells to be analyzed are or comprise monocytes.

In these embodiments, the TIRC7 polynucleotides, nucleic acid molecules, (poly)peptide, antibodies or ligands preferably labeled with a detectable moiety. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Commonly used labels comprise, inter alia, fluorochromes (like fluorescein, rhodamine, Texas Red, etc.), enzymes (like horse radish peroxidase, β-galactosidase, alkaline phosphatase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, random priming, nick-translations, tailing (using terminal transferases) are well known in the art. Detection methods comprise, but are not limited to, autoradiography, fluorescence microscopy, direct and indirect enzymatic reactions, etc.

In addition, the above-described compounds etc. may be attached to a solid phase. Solid phases are known to those in the art and may comprise polystyrene beads, latex beads, magnetic beads, colloid metal particles, glass and/or silicon chips and surfaces, nitrocellulose strips, membranes, sheets, animal red blood cells, or red blood cell ghosts, duracytes and the walls of wells of a reaction tray, plastic tubes or other test tubes. Suitable methods of immobilizing TIRC7 nucleic acids, (poly)peptides, proteins, antibodies, etc. on solid phases include but are not limited to ionic, hydrophobic, covalent interactions and the like. The solid phase can retain one or more additional receptor(s) which has/have the ability to attract and immobilize the region as defined above. This receptor can comprise a charged substance that is oppositely charged with respect to the reagent itself or to a charged substance conjugated to the capture reagent or the receptor can be any specific binding partner which is immobilized upon (attached to) the solid phase and which is able to immobilize the reagent as defined above.

Commonly used detection assays can comprise radioisotopic or non-radioisotopic methods. These comprise, inter alia, RIA (Radioisotopic Assay) and IRMA (Immune Radioimmunometric Assay), EIA (Enzym Immuno Assay), ELISA (Enzyme Linked Immuno Assay), FIA (Fluorescent Immuno Assay), and CLIA (Chemioluminescent Immune Assay). Other detection methods that are used in the art are those that do not utilize tracer molecules. One prototype of these methods is the agglutination assay, based on the property of a given molecule to bridge at least two particles.

For diagnosis and quantification of (poly)peptides, polynucleotides, etc. in clinical and/or scientific specimens, a variety of immunological methods, as described above as well as molecular biological methods, like nucleic acid hybridization assays, PCR assays or DNA Enzyme Immunoassays (Mantero et al., Clinical Chemistry 37 (1991), 422-429) have been developed and are well known in the art. In this context, it should be noted that the TIRC7 nucleic acid molecules may also comprise PNAs, modified DNA analogs containing amide backbone linkages. Such PNAs are useful, inter alia, as probes for DNA/RNA hybridization.

The above-described compositions may be used for methods for detecting expression of a TIRC7 polynucleotide by detecting the presence of mRNA coding for a TIRC7 (poly)peptide which comprises, for example, obtaining mRNA from cells of a subject and contacting the mRNA so obtained with a probe/primer comprising a nucleic acid molecule capable of specifically hybridizing with a TIRC7 polynucleotide under suitable hybridization conditions, and detecting the presence of mRNA hybridized to the probe/primer. Further diagnostic methods leading to the detection of nucleic acid molecules in a sample comprise, e.g., polymerase chain reaction (PCR), ligase chain reaction (LCR), Southern blotting in combination with nucleic acid hybridization, comparative genome hybridization (CGH) or representative difference analysis (RDA). These methods for assaying for the presence of nucleic acid molecules are known in the art and can be carried out without any undue experimentation.

The present invention also relates to a kit for use in any one of the above described methods, said kit comprising an anti-TIRC7 antibody or TIRC7 antisense nucleic acid molecule, or a derivative thereof Such kits are used to detect DNA which hybridizes to TIRC7 DNA or to detect the presence of TIRC7 protein or peptide fragments in a sample. Such characterization is useful for a variety of purposes including but not limited to forensic analyses, diagnostic applications, and epidemiological studies in accordance with the above-described methods of the present invention. The recombinant TIRC7 proteins, DNA molecules, RNA molecules and antibodies lend themselves to the formulation of kits suitable for the detection and typing of TIRC7. Such a kit would typically comprise a compartmentalized carrier suitable to hold in close confinement at least one container. The carrier would further comprise reagents such as recombinant TIRC7 protein or anti-TIRC7 antibodies suitable for detecting TIRC7. The carrier may also contain a means for detection such as labeled antigen or enzyme substrates or the like.

In addition, the present invention also relates to a method of identifying or isolating a therapeutic agent capable of modulating increase or decrease in phagocytosis and/or monocyte population or increasing lymphocyte response to antigens in a subject comprising a screening method for antagonists/inhibitors or agonist/activators of TIRC7. Generally, screening methods for antagonists/inhibitors or agonist/activators of TIRC7 are described in WO99/11782 and in PCT/EP01/12485.

Preferably, any one of the above described diagnostic methods, screening methods and kits are used in the detection or screening of disorders related to phagocytosis and/or lymphocyte activity, most preferably those described above.

In a further aspect, the present invention relates to a method to produce an immunoglobulin or an analog thereof, specific for a desired antigen, which comprises:
(a) administering said antigen or an immunogenic portion thereof to a nonhuman animal under conditions to stimulate an immune response, whereby said animal produces B cells that secrete immunoglobulin specific for said antigen; wherein said nonhuman animal is characterized by being substantially incapable of producing endogenous T-cell immune response cDNA 7 (TIRC7) or TIRC7 activity in lymphocytes; and
(b) recovering said immunoglobulin or analog.

This aspect of the invention is based on the surprising finding that B cell proliferation as well as immunoglobulin expression were induced in TIRC7 (−/−) mice splenocytes following activation with IL-4 and LPS; see examples 6 and 7. Thus, nonhuman animals wherein the activity of TIRC7 has been substantially reduced in the appropriate cells, preferably at least in B cells, for example by knock out or antisense approaches can advantageously be used for antibody production. Alternatively, the normal immunization process is accompanied by administering an antagonist/inhibitor of TIRC7 in order to exogenously bring about the same effect as observed with the TIRC7 (−/−) mice in the examples. Hence, in principle any known method for the production of monoclonal antibodies may be used except that in addition or alternatively TIRC7 activity is substantially reduced in at least some if not all of the cells of the nonhuman animal which has been immunized with a desired antigen. Preferably, TIRC7 activity is substantially reduced in at least the lymphocytes of the nonhuman animal, at least at some stage of the immunization process. For production of the desired antibodies, the first step is administration of the antigen. Techniques for such administration are conventional and involve suitable immunization protocols and formulations which will depend on the nature of the antigen per se. It may be necessary to provide the antigen with a carrier to enhance its immunogenicity and/or to include formulations which contain adjuvants and/or to administer multiple injections and/or to vary the route of the immunization, and the like. Such techniques are standard and optimization of them will depend on the characteristics of the particular antigen for which immunospecific reagents are desired. Such methods including methods of immunization to enhance the immune response to specific antigens in vivo are well known in the art and are described for example in Rudbach, Methods Mol. Biol. 45 (1995), 1-8 and Dean, Methods Mol. Biol. 80 (1998), 23-37. The method of the present invention also encompasses methods to produce human antibodies such as described in WO96/33735 with the mentioned modifications. As mentioned before, the effect of reducing TIRC7 activity may be achieved by means other that inactivating the TIRC7 gene. Thus, in one embodiment the antigen or an immunogenic portion thereof is administered in conjunction with an TIRC7 antagonist as described in the afore mentioned embodiments to the nonhuman animal.

As used herein, the term “immunospecific reagents” includes immunoglobulins and their analogs. The term “analogs” has a specific meaning in this context. It refers to moieties that contain the immunoglobulin which account for its immunospecificity. In particular, complementarity determining regions (CDRs) are required, along with sufficient portions of the framework (FRs) to result in the appropriate three dimensional conformation. The person skilled in the art knows that each variable domain (the heavy chain V_{H} and light chain V_{L}) Of an antibody comprises three hypervariable regions, sometimes called complementarity determining regions or “CDRs” flanked by four relatively conserved framework regions or “FRs”. The CDRs contained in the variable regions of the antibody of the invention can be determined, e.g., according to Kabat, Sequences of Proteins of Immunological Interest (U.S. Department of Health and Human Services, third edition, 1983, fourth edition, 1987, fifth edition 1990 and updated ones). Typical immunospecific analogs of antibodies include F(ab″)2, Fab′, and Fab regions. Modified forms of the variable regions to obtain, for example, single chain Fv analogs with the appropriate immunospecificity are known. A review of such Fv construction is found, for example, in Huston, Methods in Enzvmology 203 (1991), 46-63. The construction of antibody analogs with multiple immunospecificities is also possible by coupling the variable regions from one antibody to those of second antibody.

The variable regions can also be coupled to a variety of additional substances which can provide toxicity, biological functionality, alternative binding specificities and the like. The moieties including the variable regions produced by the methods of the invention include single-chain fusion proteins, molecules coupled by covalent methods other than those involving peptide linkages, and aggregated molecules. Examples of analogs which include variable regions coupled to additional molecules covalently or noncovalently include those in the following nonlimiting illustrative list. Traunecker, Int. J. Cancer Surp. SuDP 7 (1992), 51-52, describe the bispecific reagent janusin in which the Fv region directed to CD3 is coupled to soluble CD4 or to other ligands such as OVCA and IL-7. Similarly, the variable regions produced by the method of the invention can be constructed into Fv molecules and coupled to alternative ligands such as those illustrated in the cited article. Higgins, J. Infect Disease 166 (1992), 198-202, described a heteroconjugate antibody composed of OKT3 cross-linked to an antibody directed to a specific sequence in the V3 region of GP120. Such heteroconjugate antibodies can also be constructed using at least the variable regions contained in the immunoglobulins produced by the invention methods. Additional examples of specific antibodies include those described by Fanger, Cancer Treat. Res. 68 (1993), 181-194 and by Fanger, Crit. Rev. Immunol. 12 (1992), 101-124. Conjugates that are immunotoxins including conventional antibodies have been widely described in the art. The toxins may be coupled to the antibodies by conventional coupling techniques or immunotoxins containing protein toxin portions can be produced as fusion proteins. The analogs of the present invention can be used in a corresponding way to obtain such immunotoxins. Illustrative of such immunotoxins are those described by Byers, Seminars Cell. Biol. 2 (1991), 59-70 and by Fanger, Immunol. Today 12 (1991), 51-54.

It will also be noted that some of the immunoglobulins and analogs of the invention will have agonist activity with respect to antigens for which they are immunospecific in the cases wherein the antigens perform signal transducing functions. Thus, a subset of antibodies or analogs prepared according to the methods of the invention which are immunospecific for, for example, a cell surface receptor, will be capable of eliciting a response from cells bearing this receptor corresponding to that elicited by the native ligand. Furthermore, antibodies or analogs which are immunospecific for substances mimicking transition states of chemical reactions will have catalytic activity. Hence, a subset of the antibodies and analogs of the invention will function as catalytic antibodies.

Naturally, the method of the present invention can further comprise recovering said polyclonal immunoglobulin or analog from said animal. Furthermore, the method of the invention may further comprise immortalizing B cells from said animal immunized with said antigen, screening the resulting immortalized cells for the secretion of said immunoglobulin specific for said antigen, and
(i) recovering immunoglobulin secreted by said immortalized B cells, or
(ii) recovering the genes encoding at least the immunoglobulin from the immortalized B cells, and optionally modifying said genes;
(iii) expressing said genes or modified forms thereof to produce the immunoglobulin or analog; and
(iv) recovering said immunoglobulin or analog.

In short, the genes encoding the immunoglobulins produced by the transgenic animals of the invention can be retrieved and the nucleotide sequences encoding the variable region can be manipulated according to known techniques to provide a variety of analogs such as those described above. In addition, the immunoglobulins themselves containing the variable regions can be modified using standard coupling techniques to provide conjugates retaining immunospecific regions.

Thus, immunoglobulin “analogs” refers to the moieties which contain those portions of the antibodies of the invention which retain their immunospecificity. These will retain sufficient variable regions to provide the desired specificity.

As stated above, all of the methods of the invention include administering the appropriate antigen to the transgenic animal. The recovery or production of the antibodies themselves can be achieved in various ways.

First, and most straightforward, the polyclonal antibodies produced by the animal and secreted into the bloodstream can be recovered using known techniques. Purified forms of these antibodies can, of course, be readily prepared by standard purification techniques, preferably including affinity chromatography with Protein A, antiimmunoglobulin, or the antigen itself. In any case, in order to monitor the success of immunization, the antibody levels with respect to the antigen in serum will be monitored using standard techniques such as ELISA, RIA and the like.

For some applications only the variable regions of the antibodies are required, which can be obtained by treating the polyclonal antiserum with suitable reagents so as to generate Fab′, Fab, or F(ab″)2 portions. Such fragments are sufficient for use, for example, in immunodiagnostic procedures involving coupling the immunospecific portions of immunoglobulins to detecting reagents such as radioisotopes.

Alternatively, immunoglobulins and analogs with desired characteristics can be generated from immortalized B cells derived from the transgenic animals used in the method of the invention or from the rearranged genes provided by these animals in response to immunization. Thus, as an alternative to harvesting the antibodies directly from the animal, the B cells can be obtained, typically from the spleen, but also, if desired, from the peripheral blood lymphocytes or lymph nodes and immortalized using any of a variety of techniques, most commonly using the fusion methods described by Kohler and Milstein Nature 245 (1975), 495. The resulting hybridomas (or otherwise immortalized B cells) can then be cultured as single colonies and screened for secretion of antibodies of the desired specificity. After the appropriate hybridomas are selected, the desired antibodies can be recovered, again using conventional techniques. They can be prepared in quantity by culturing the immortalized B cells using conventional methods, either in vitro or in vivo to produce ascites fluid. Purification of the resulting monoclonal antibody preparations is less burdensome than in the case of serum since each immortalized colony will secrete only a single type of antibody. In any event, standard purification techniques to isolate the antibody from other proteins in the culture medium can be employed.

As an alternative to obtaining immunoglobulins directly from the culture of immortalized B cells derived from the animal, the immortalized cells can be used as a source of rearranged heavy chain and light chain loci for subsequent expression and/or genetic manipulation. Rearranged antibody genes can be reverse transcribed from appropriate mRNAs to produce cDNA. If desired, the heavy chain constant region can be exchanged for that of a different isotype or eliminated altogether. The variable regions can be linked to encode single chain Fv regions. Multiple Fv regions can be linked to confer binding ability to more than one target or chimeric heavy and light chain combinations can be employed. Once the genetic material is available, design of analogs as described above which retain their ability to bind the desired target is straightforward.

Once the appropriate genetic material is obtained and, if desired, modified to encode an analog, the coding sequences, including those that encode, at a minimum, the variable regions of the heavy and light chain, can be inserted into expression systems contained on vectors which can be transfected into standard recombinant host cells. A variety of such host cells may be used; for efficient processing, however, mammalian cells are preferred. Typical mammalian cell lines useful for this purpose include CHO cells, 293 cells, or NSO cells. The production of the antibody or analog is then undertaken by culturing the modified recombinant host under culture conditions appropriate for the growth of the host cells and the expression of the coding sequences. The antibodies are then recovered from the culture. The expression systems are preferably designed to include signal peptides so that the resulting antibodies are secreted into the medium; however, intracellular production is also possible.

In addition to deliberate design of modified forms of the immunoglobulin genes to produce analogs, advantage can be taken of phage display techniques to provide libraries containing a repertoire of antibodies with varying affinities for the desired antigen. For production of such repertoires, it is unnecessary to immortalize the B cells from the immunized animal; rather, the primary B cells can be used directly as a source of DNA. The mixture of cDNAs obtained from B cells, e.g., derived from spleens, is used to prepare an expression library, for example, a phage display library transfected into E. coli. The resulting cells are tested for immunoreactivity to the desired antigen. Techniques for the identification of high affinity antibodies from such libraries are described by Griffiths, EMBO J. 13 (1994), 3245-3260; Nissim, ibid, 692-698, and Griffiths, ibid, 12, 725-734. Ultimately, clones from the library are identified which produce binding affinities of a desired magnitude for the antigen, and the DNA encoding the product responsible for such binding is recovered and manipulated for standard recombinant expression. Phage display libraries may also be constructed using previously manipulated nucleotide sequences and screened in similar fashion. In general, the cDNAs encoding heavy and light chain are independently supplied or are linked to form Fv analogs for production in the phage library. The phage library is then screened for the antibodies with highest affinity for the antigen and the genetic material recovered from the appropriate clone.

Further rounds of screening can increase the affinity of the original antibody isolated. The manipulations described above for recombinant production of the antibody or modification to form a desired analog can then be employed.

There are large numbers of antigens for which antibodies and their analogs would be made available by the methods of the invention. These include, but are not limited to, the following nonlimiting set: leukocyte markers, such as CD2, CD3, CD4, CD5, CD6, CD7, CD8, CD11a,b,c, CD13, CD14, CD18, CD19, CD20, CD22, CD23, CD27 and its ligand, CD28 and its ligands B7.1, B7.2, B7.3, CD29 and its ligand, CD30 and its ligand, CD40 and its ligand gp39, CD44, CD45 and isoforms, Cdw52 (Campath antigen), CD56, CD58, CD69, CD72, CTLA-4, LFA-1 and TCR; histocompatibility antigens, such as MMC class I or II, the Lewis Y antigens, Slex, Sley, Slea, and Selb;

adhesion molecules, including the integrins, such as VLA-1, VLA-2, VLA-3, VLA-4, VLA-5, VLA-6, LFA-1, Mac-1, amp3, and p150,95; and

the selectins, such as L-selectin, E-selectin, and P-selectin and their counterreceptors VCAM-1, ICAM-1, ICAM-2, and LFA-3;

interleukins, such as IL-1, IL-2, IL-3, IL-4, IL-5, IL-6, IL-7, IL-8, IL-9, IL-10, IL-11, IL-12, IL-13, IL-14, and IL-15;

interleukin receptors, such as IL-1R, IL-2R, IL-3R, IL-4R, IL-5R, IL-6R, IL-7R, IL-8R, IL-9R, IL-10R, IL-11R, IL-12R, IL-13R, IL-14R and IL-15R;

chemokines, such as PF4, RANTES, MIP1a, MCP1, IP10, ENA-78, NAP-2, Groa, Grow, and IL-8;

growth factors, such as TNFalpha, TGFbeta, TSH, VEGF/VPF, PTHrP, EGF family, FGF, PDGF family, endothelin, Fibrosin (FsF,), Laminin, and gastrin releasing peptide (GRP);

growth factor receptors, such as TNFalphaR, RGFbetaR, TSHR, VEGFR/VPFR, FGFR, EGFR, PTHrPR, PDGFR family, EPO-R, GCSF-R and other hematopoietic receptors;

interferon receptors, such as IFNaR, IFNPR, and IFNyR;

Igs and their receptors, such as IGE, FceRI, and FceRII;

tumor antigens, such as her2-neu, mucin, CEA and endosialin;

allergens, such as house dust mite antigen, lol pl (grass) antigens, and urushiol;

viral proteins, such as CMV glycoproteins B, H, and gCIII, HIV-1 envelope glycoproteins, RSV envelope glycoproteins, HSV envelope glycoproteins, EBV envelope glycoproteins, VZV, envelope glycoproteins, HPV envelope glycoproteins, Hepatitis family surface antigens;

toxins, such as pseudomonas endotoxin and osteopontin/uropontin, snake venom, spider venom, and bee venom;

blood factors, such as complement C3b, complement C5a, complement C5b-9, Rh factor, fibrinogen, fibrin, and myelin associated growth inhibitor;

enzymes, such as cholesterol ester transfer protein, membrane bound matrix metalloproteases, and glutamic acid decarboxylase (GAD); and

miscellaneous antigens including ganglioside GD3, ganglioside GM2, LMP1, LMP2, eosinophil major basic protein, PTHrp, eosinophil cationic protein, pANCA, Amadori protein, Type IV collagen, glycated lipids, v-interferon, A7, Pglycoprotein and Fas (AFO-1) and oxidized-LDL.

As mentioned before, the immunoglobulin or its encoding cDNAs may be further modified. Thus, in a further embodiment the method of the present invention comprises any one of the step(s) of producing a chimeric antibody, humanized antibody, single-chain antibody, Fab-fragment, bi-specific antibody, fusion antibody, labeled antibody or an analog of any one of those. Corresponding methods are known to the person skilled in the art and are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to the same epitope as that of any one of the antibodies described herein (Schier, Human Antibodies Hybridomas 7 (1996), 97-105; Malmborg, J. Immunol. Methods 183 (1995), 7-13). The production of chimeric antibodies is described, for example, in WO89/09622. Methods for the production of humanized antibodies are described in, e.g., EP-A1 0 239 400 and WO90/07861. A further source of antibodies to be utilized in accordance with the present invention are so-called xenogeneic antibodies. The general principle for the production of xenogeneic antibodies such as human antibodies in mice is described in, e.g., WO 91/10741, WO 94/02602, WO 96/34096 and WO 96/33735. As discussed above, the antibody of the invention may exist in a variety of forms besides complete antibodies; including, for example, Fv, Fab and F(ab)2, as well as in single chains; see e.g. WO88/09344. The antibodies of the present invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) N.Y. and Ausubel, Current Protocols in Molecular Biology, Green Publishing Associates and Wiley Interscience, N.Y. (1994). Modifications of the antibody of the invention include chemical and/or enzymatic derivatizations at one or more constituent amino acid, including side chain modifications, backbone modifications, and N— and C-terminal modifications including acetylation, hydroxylation, methylation, amidation, and the attachment of carbohydrate or lipid moieties, cofactors, and the like. Likewise, the present invention encompasses the production of chimeric proteins which comprise the described antibody or some fragment thereof at the amino terminus fused to heterologous molecule such as an immunostimulatory ligand at the carboxyl terminus; see, e.g., WO00/30680 for corresponding technical details.

For therapeutic applications, the antibodies may be administered in a pharmaceutically acceptable dosage form. They may be administered by any means that enables the active agent to reach the desired site of action, for example, intravenously as by bolus or by continuous infusion over a period of time, by intramuscular, subcutaneous, intraarticular, intrasynovial, intrathecal, oral, topical or inhalation routes. The antibodies may be administered as a single dose or a series of treatments. For parenteral administration, the antibodies may be formulated as a solution, suspension, emulsion or lyophilized powder in association with a pharmaceutically acceptable parenteral vehicle. If the antibody is suitable for oral administration, the formulation may contain suitable additives such as, for example, starch, cellulose, silica, various sugars, magnesium carbonate, or calcium phosphate. Suitable vehicles are described in the most recent edition of Remington's Pharmaceutical Sciences, a standard reference text in this field.

For prevention or treatment of disease, the appropriate dosage of antibody will depend upon known factors such as the pharmacodynamic characteristics of the particular antibody, its mode and route of administration, the age, weight, and health of the recipient, the type of condition to be treated and the severity and course of the condition, frequency of treatment, concurrent treatment and the physiological effect desired.

In a still further embodiment, the present invention relates to a vaccine comprising a TIRC7 antagonist/inhibitor such as any one of those described above. This embodiment is based on the surprising finding that TIRC7 deficient mice exhibit increased T and B cell proliferative response to different stimuli in vitro and in vivo compared with wild type littermates; see examples 4 to 7. In particular, Type-1 immune response was shown to be pronounced. Accordingly, the invention provides means and methods towards the rational design of Th1 adjuvants such as those discussed in Moingeon, Vaccine 19 (2001), 4363-4372. Methods how to formulate and administer TIRC7 antagonists/inhibitors as vaccine components can be derived from the prior art; see for example Ragupathi et al. in Vaccine 19 (2000), 530-537, which describe the effect of immunological adjuvant combinations on the antibody and T-cell response to vaccination with MUC1-KLH and GD3-KLH conjugates. Hence, in a similar fashion TIRC7 antagonists/inhibitors can be used to augment antibody and T-cell responses against vaccines containing a desired antigen. Another example in this respect is the use of interleukin 12 to enhance the cellular immune response of swine to an inactivated herpesvirus vaccine; see Zuckermann, Adv. Vet. Med. 41 (1999), 447-461. Accordingly, the present invention relates to the use of any one of the above described TIRC7 antagonists/inhibitors as an adjuvant.

This invention will be better understood by reference to the Examples which follow, but those skilled in the art will readily appreciate that they are only illustrative of the invention as described more fully in the claims which follow thereafter. In addition, various documents are cited throughout this application. The disclosures of these documents (including any manufacturer's specifications, instructions, etc.) are hereby incorporated by reference into this application to describe more fully the state of the art to which this invention pertains; however, there is no admission that any document cited is indeed prior art as to the present invention.

### EXAMPLES

### Example 1

### Generation of TIRC7 Deficient Mice

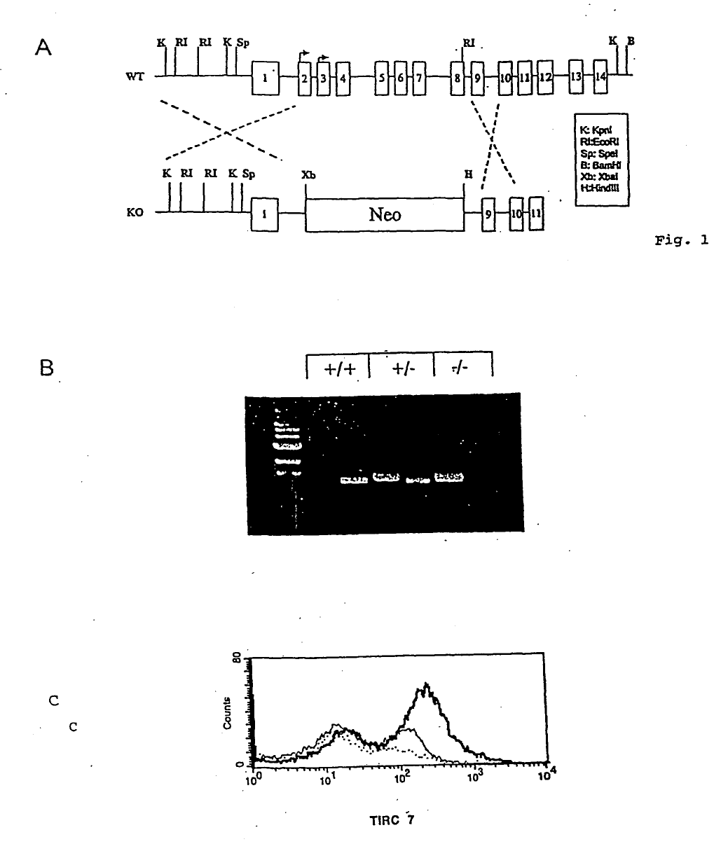

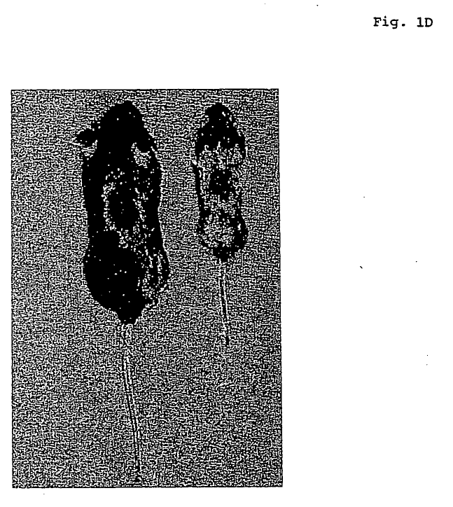

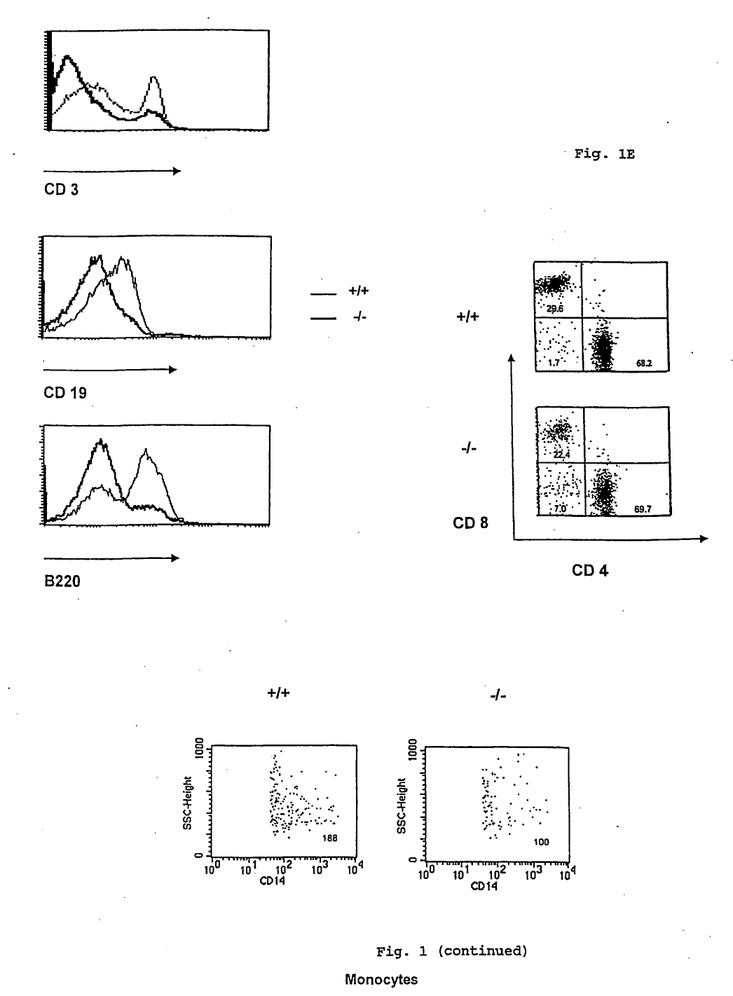

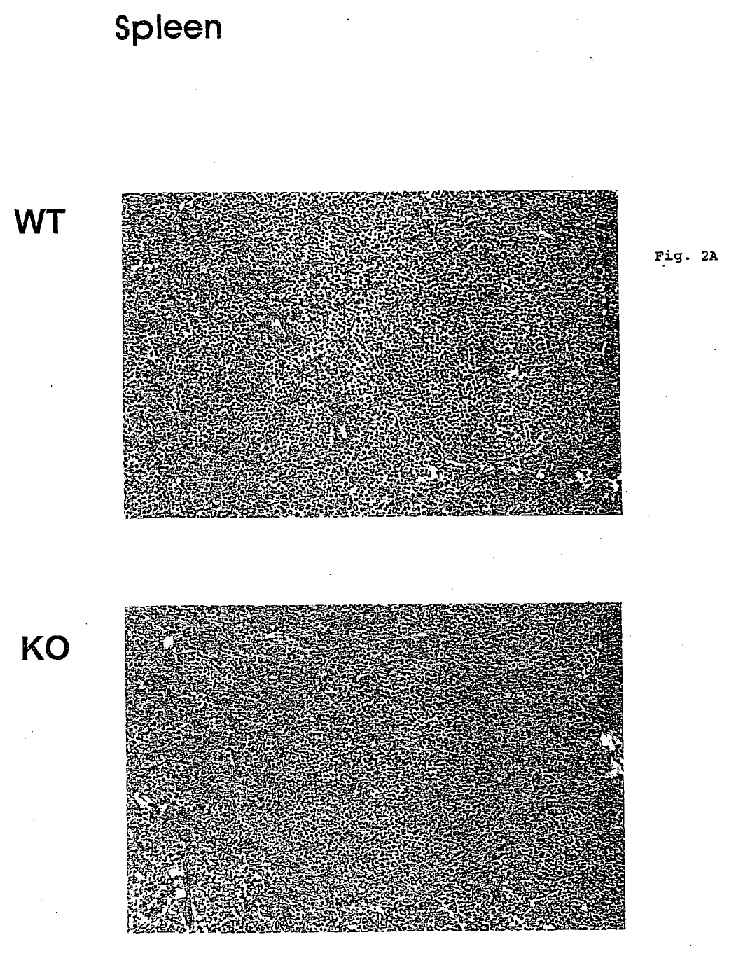

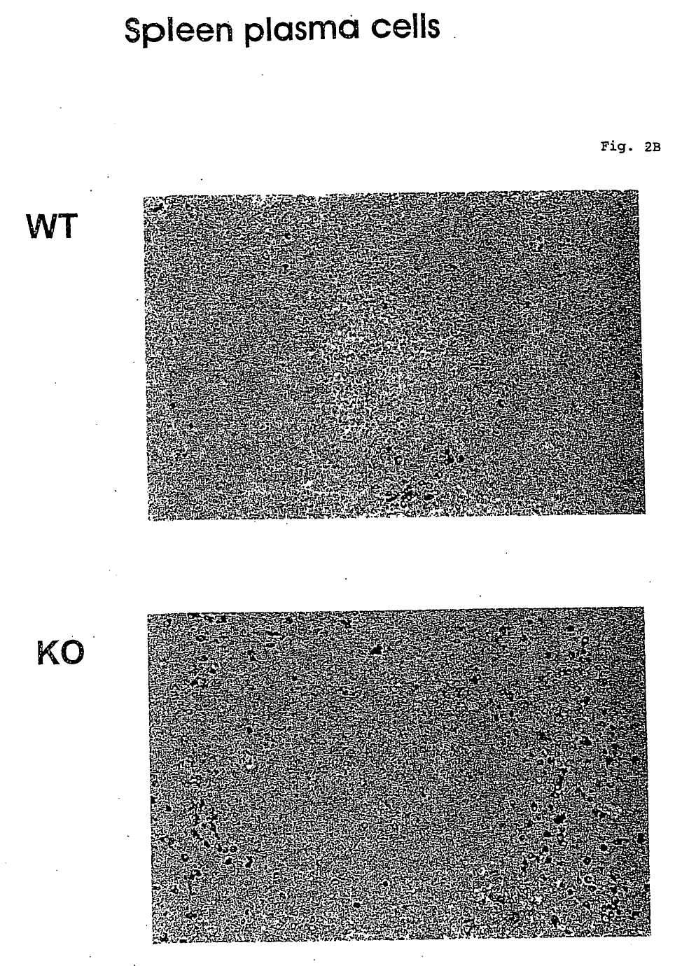

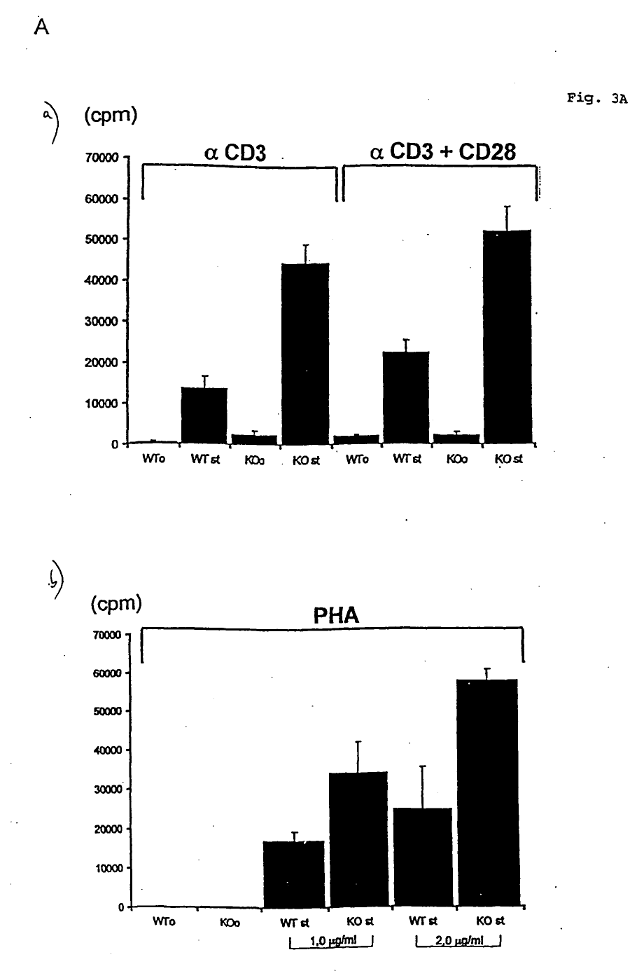

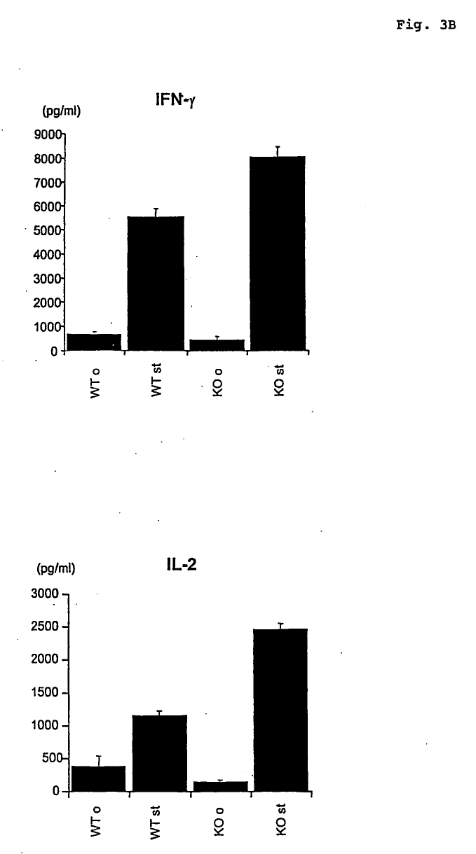

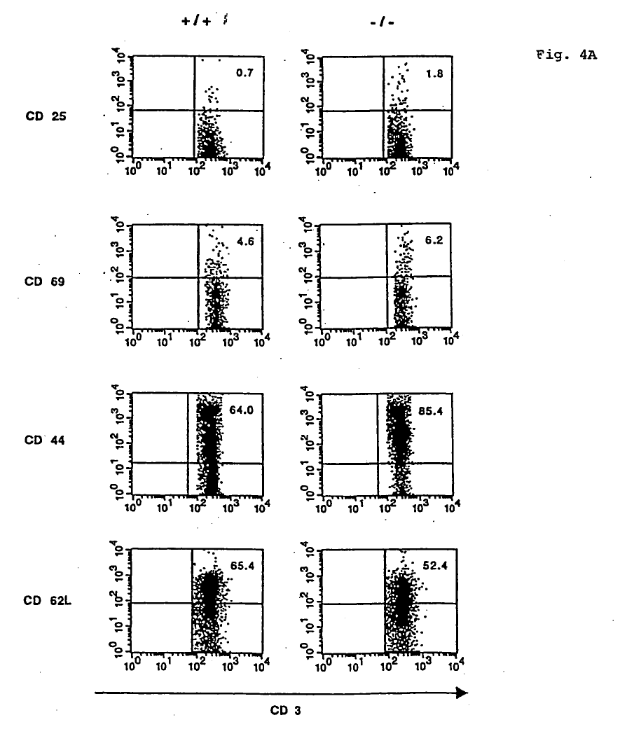

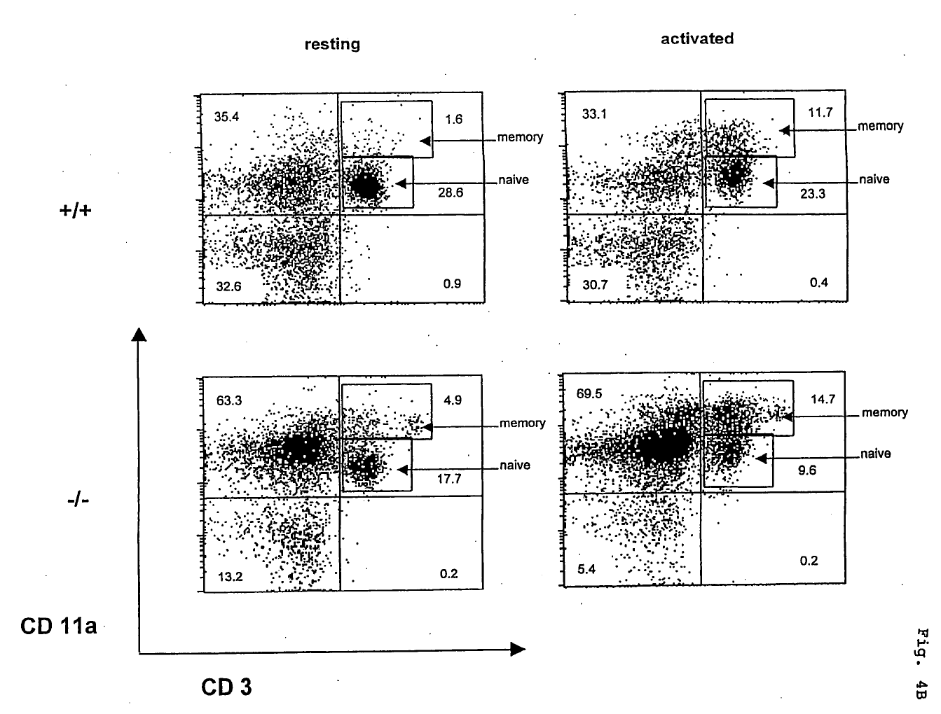

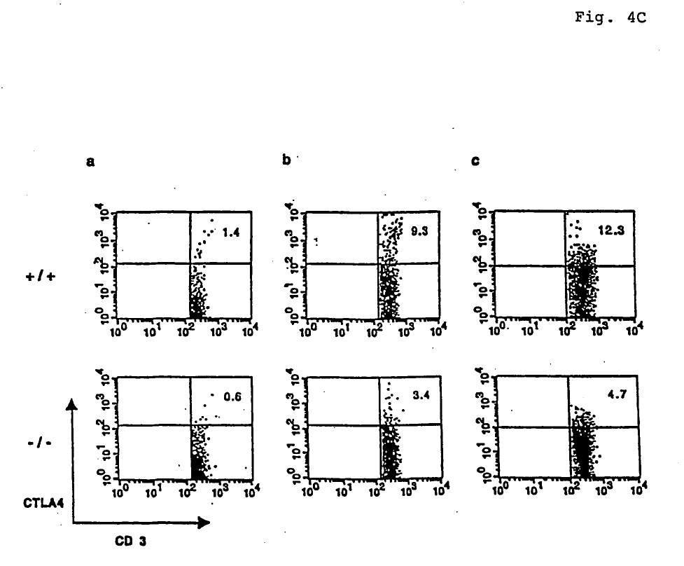

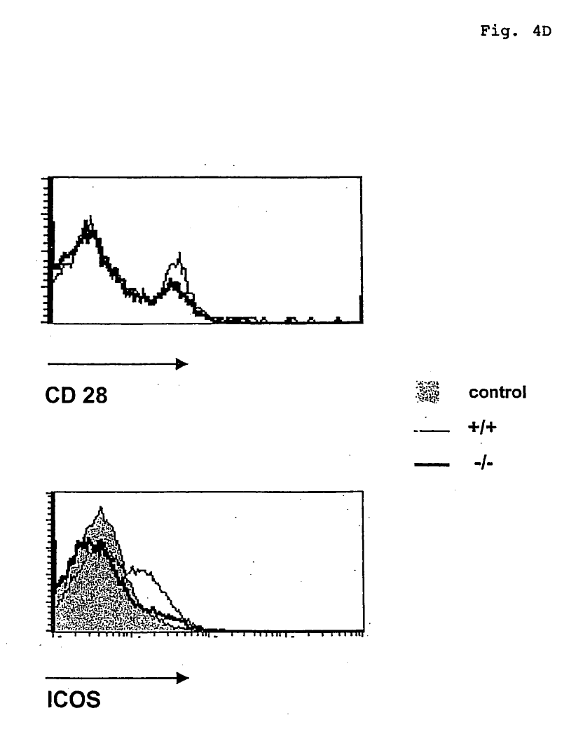

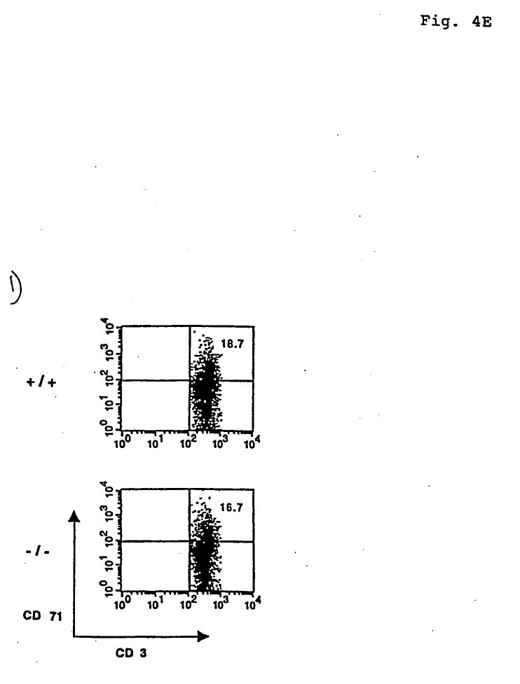

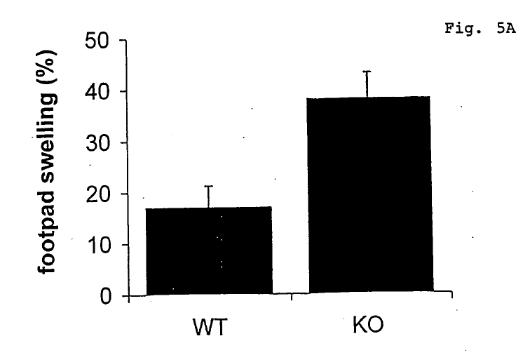

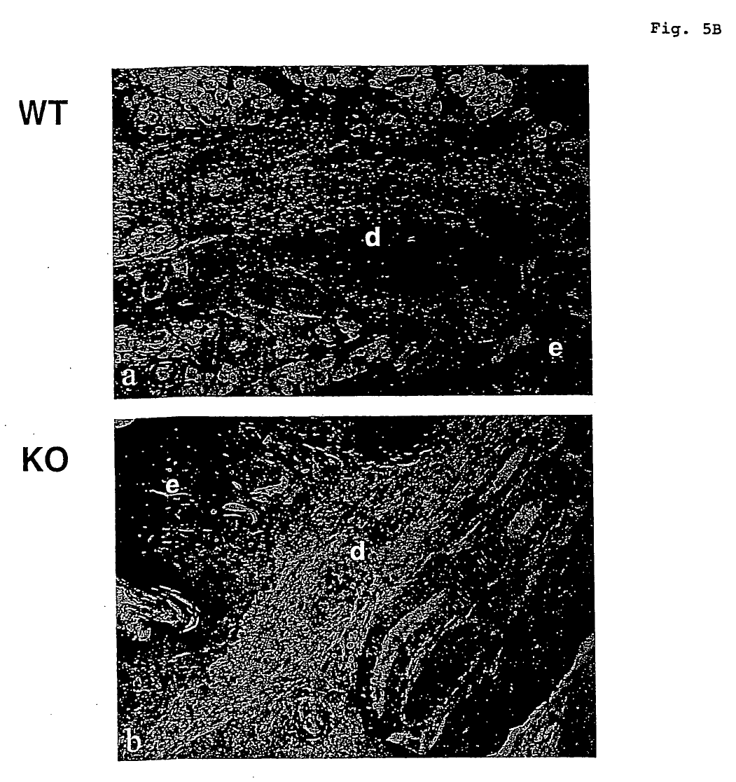

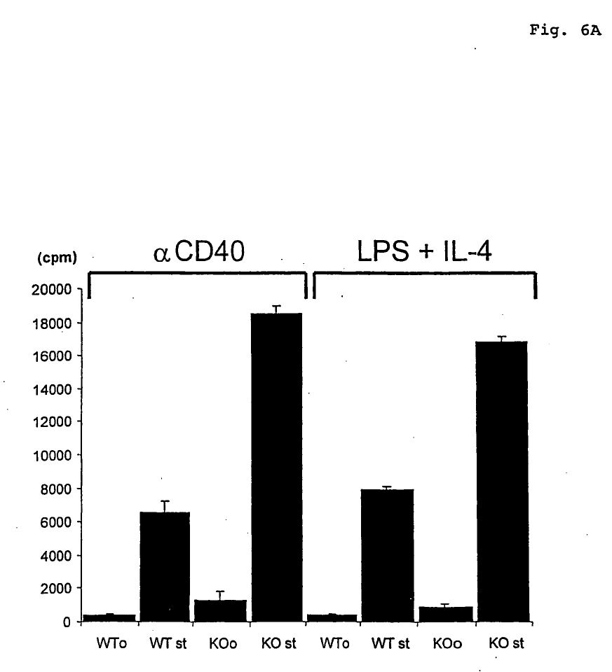

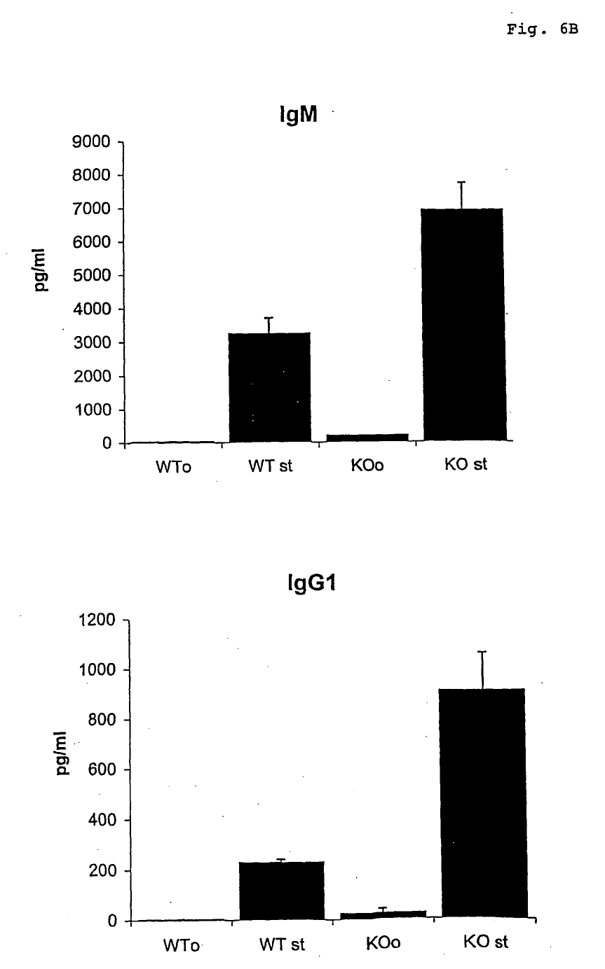

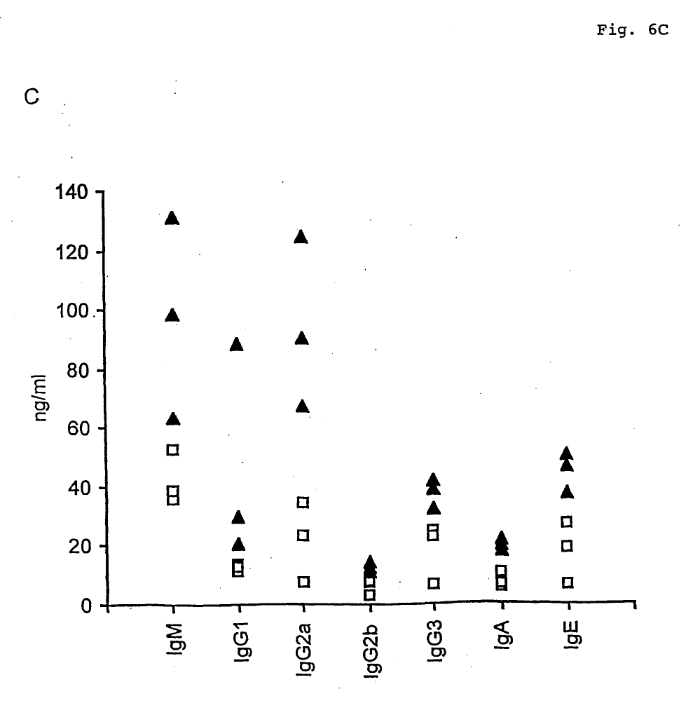

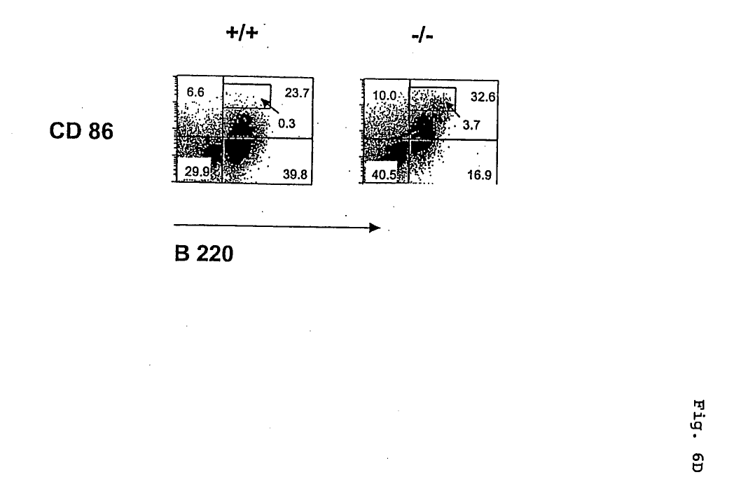

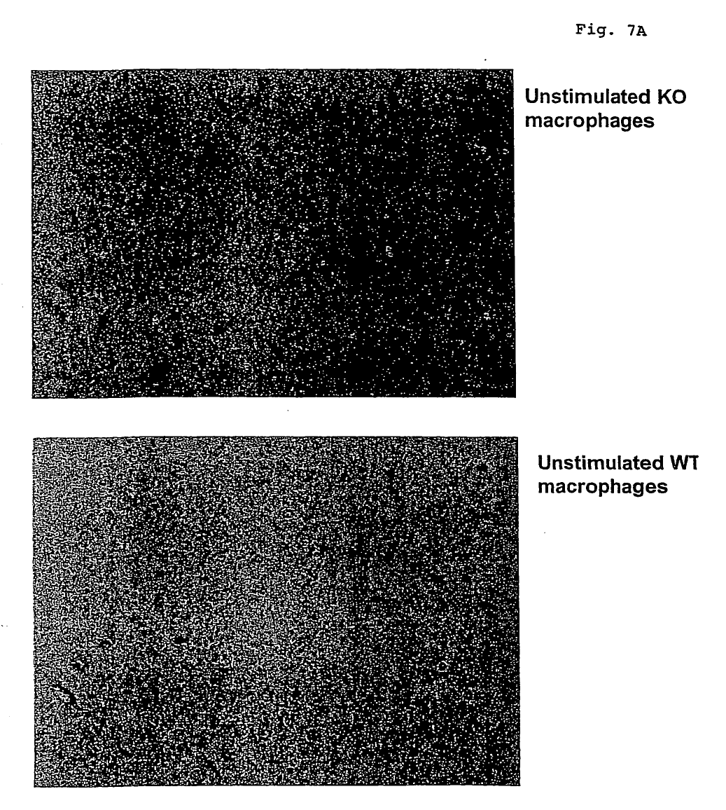

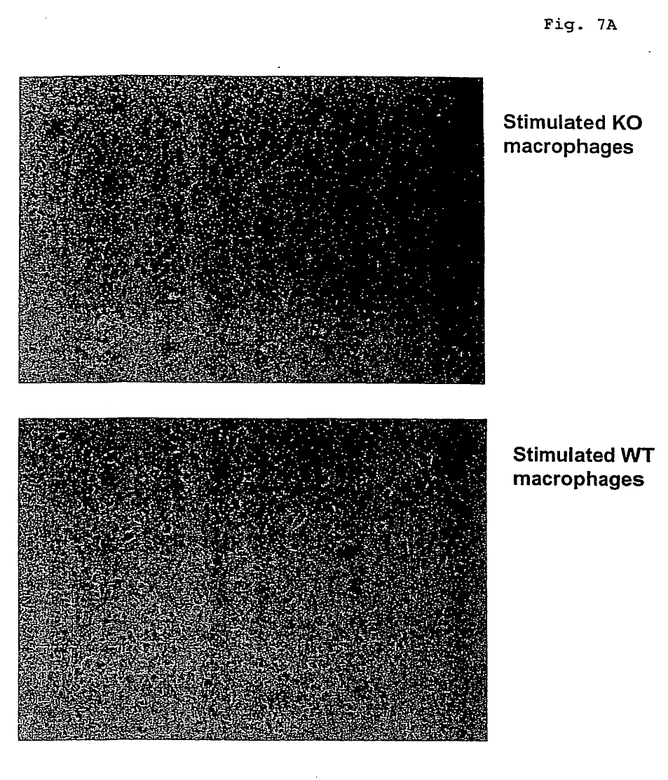

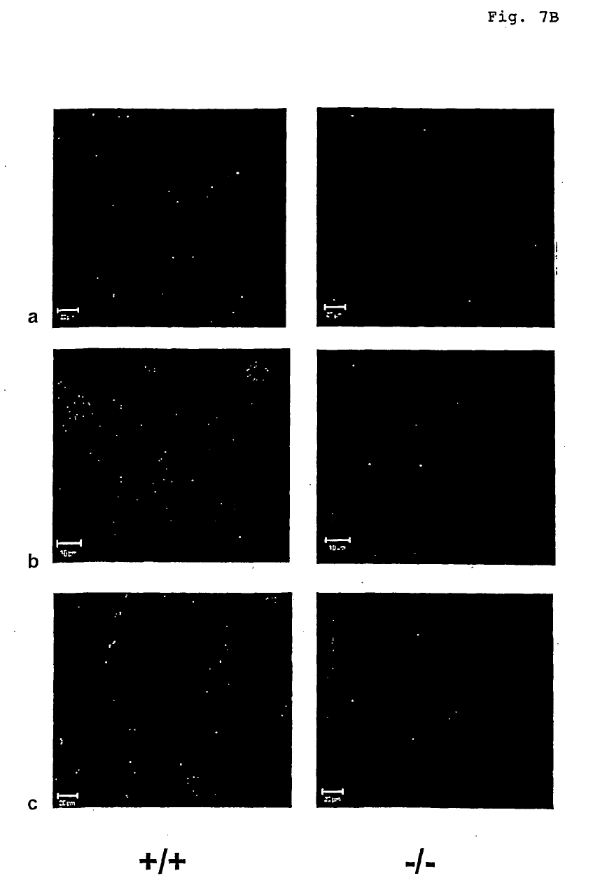

To characterize the functional importance of TIRC7, mice were generated in which the TIRC7 locus was disrupted by homologous gene targeting (Tivol, Immunity 3 (1995), 541-547). A targeting vector was constructed which was used to replace sequences encoding the exons 2-8 of TIRC7 with the neomycin drug resistance gene (FIG. 1A) (Tivol et al., 1995). Gene targeting was performed in C57 black mice in accordance with established protocols (Capecchi, Science 244 (1989), 1288-1292). In ES cells derived from strain 129 mouse embryos a 2 kb genomic fragment of exon 2 to 8 of TIRC7 was replaced by insertion of a cassette containing the neomycin resistance gene. Transfection and culturing of the ES cells was performed as described by Forster et al., (Cell 87 (1996), 1037-1047). Chimeric males were mated with C57 females and genotype of the offspring were determined by PCR of tail DNA using oligonucleotide primers detecting the neomycin cassette, exon 9 and exon 11, in addition to Southern blotting. Genotyping of the progeny resulting from an inter cross of two animals heterozygous for the disrupted TIRC7 gene locus was performed as demonstrated by PCR of genomic DNA using TIRC7 specific primers (FIG. 1B). Homozygous mutant offspring were produced in a typical Mendelian frequency. The lack of TIRC7 protein on CD3 positive T cells of homozygous offsprings was confirmed by staining with anti-TIRC7 antibody and subsequent FACS analysis (FIG. 1C). The phenotype of TIRC7 deficient mice demonstrated significantly reduced body weight compared with wild type littermates (FIG. 1D).

### Example 2

### TIRC7 Deficient Mice Exhibits Decrease of All Mononuclear Cell Populations

To examine whether an absence of TIRC7 expression affects development of cell populations within lymphoid organs flow cytometric analyses were performed of single cell suspensions from splenocytes obtained from TIRC7 deficient and WT littermates (FIG. 1E). Also a significant decrease of monocytes in mice lacking TIRC7 protein was observed in comparison to WT mice (FIG. 1F).

Single cell suspensions of mouse spleens and lymph nodes were prepared by grinding tissue through a sterile wire mesh and passing through a 50 mm filter. All procedures were carried out under sterile conditions in RPMI 1640 medium (Biochom KG) supplemented with 10% fetal calf serum (Biochom KG), 5 mM glutamine, penicillin and streptomycin (Gibco BRL). Cells were stained for 30 mins at 4° C. in 100 μl of PBS and then washed prior to analysis. FACS analysis was performed as described by Waldrop et al. (JCI 99 (1997),1739-1750). Cells were stained with a panel of fluorochrome-conjugated antibodies, including FITC labeled anti-CD3 mAb and anti-CD19 mAb, PE labeled anti-CTLA4, anti-CD3, anti-CD28, anti-CD25, anti-CD69, anti-CD44, anti-CD62L, anti-CD11a, anti-CD71 and anti-CD86 monoclonal antibodies. PerCP labeled anti-B220 and APC labeled anti-CD4 mAb and anti-CD8 mAb antibodies were purchased from PharMingen. Anti-ICOS antibody was purchased from Santa Cruz Biotechnology, and donkey F(ab′)₂ anti-rabbit as well as goat F(ab′)₂ anti-mouse antibodies from Jackson Laboratories. Analyses were performed by using a FACScan flow cytometer (Becton Dickinson). Cells were analyzed using Cell Quest software (Becton Dickinson).

### Example 3

### Histological Analysis of TIRC7 Deficient Mice Revealed Atrophy of All Immune Tissues

As shown in FIG. 2A, the histological analysis of spleen isolated from TIRC7(−/−) mice and WT littermates showed a prominent hypoplasia of the splenic white pulp with disproportioned T and B cell areas. Histological methods have been used essentially as described by Karulin et al. in J. Immunol. 168 (2002), 545-553. Numerous large PALS and small B lymphocytic follicles with a lack of B cell areas were observed in spleens of TIRC7-deficient mice in comparison to WT littermates. Nevertheless, the B lymphocytic follicles of TIRC7 (−/−) spleens revealed increased numbers of germinal centers. In addition to the regressing white pulp hypoplasia of TIRC7 (−/−) cells, the splenic red pulp revealed a plasma cell hyperplasia, increased numbers of Russel bodies combined with an enhanced granulopoesis which supports the findings of an increased number of a Gr-1 positive cell fraction, described in FIG. 1F. As shown in FIG. 2B these findings were confirmed by immunohistological staining of the splenic red pulpa which demonstrate the hyperplasia of plasma cells in TIRC7 knock out mice.

Histological analysis of the thymus obtained from TIRC7 (−/−) and WT mice revealed different stages of atrophy, predominantly in the cortex. Disintegration and apoptosis of TIRC7 (−/−) thymocytes was striking compared to WT littermates. Architectural structure of peripheral lymph nodes of TIRC7 mutant mice lymphocytes in the cortex and paracortex are sparely present. Strikingly, the paracortex of peripheral lymphnodes of TIRC7 deficient mice exhibit, in contrast to WT littermates an increased number of apoptotic lymphocytes, similar to the findings within the splenic white pulp.

### Example 4

### Deletion of TIRC7 Leads to Significantly Increase of Proliferative T Cell Response and Induction of Th1 Cytokines in the Presence of Alloantigen or Mitogen

The modulation of TIRC7 signalling with specific antibody inhibited proliferation of T cells of human PBMC's (Utku et al., 1998). It was therefore assessed whether TIRC7 deficient mice are able to response to various antigens. Cell proliferation assays (assessed by incorporation of [³H] thymidine) were performed in vitro on cells isolated from spleen (FIG. 3A) and lymph node from WT and mutant mice. For T-cell proliferation assays, lymphocytes were stimulated with 10 μg/ml anti-CD3, anti-CD28 mAb (PharMingen) in pre-coated wells or with PHA (1 μg/ml or 2 μg/ml)(Sigma) at 37° C. for 48 h in 96-well plates. Cultures were then pulsed with 0.5 μCi [³H] thymidine (ICN Biochemicals) and after 18 h incubation cells were harvested and cell proliferation was determined by measuring thymidine incorporation (cpm) using a scintillation counter. As demonstrated in FIG. 3A, splenocytes from TIRC7 (−/−) mice exhibit a significantly increased proliferative response to anti-CD3 antibody alone, or together with anti-CD28 antibody compared to WT splenocytes. Similarly, following mitogenic activation of cells with PHA, a dose-dependent increase in the proliferative response of cells isolated from TIRC7(−/−) mice was observed, which substantially exceeded that observed in cells from wild type animals (FIG. 3A, a and b). 2×10⁶ cell/ml lymphocyte suspensions (from spleen or Iymph node) were incubated with 1.5 μg/ml PHA or 100 ng/ml LPS and 10 ng/ml of recombinant IL-4 (PharMingen) at 37° C. for 48 h in microtitre plates. IFN-γ and IL-2 cytokines were measured in supernatants of PHA stimulated cells. Cytokine levels were determined by ELISA using capture-, detection- and standard-antibodies obtained from PharMingen. PHA activation of splenocytes from TIRC7(−/−) mice resulted in a significant upregulation of Th1 specific cytokine expression (IL-2 and IFN-γ) compared with that of cells isolated from wild type littermates (FIG. 3B).

### Example 5

### TIRC7 Deletion Affects Expression of Several Activation Molecules and Costimulatory Molecules on T Cells

Flow cytometric analysis was performed as described in example 2. The expression analysis of lymphocyte activation markers CD69 and CD25 demonstrated only a moderate increase in resting T cells from TIRC7(−/−) mice compared with control T cells from WT littermates (FIG. 4A). Expression of CD62L and CD44, both representing marker molecules for memory and naive T cell populations, were found to be slightly decreased and elevated, respectively, compared with wild type cells (FIG. 4A). CD11a staining showed a significant increased memory cell population in T cell from TIRC7 knock out mice in comparison to the wild type littermates. Resting T cells demonstrated almost threefold more CD11a high population (4.9% in knock out and 1.6% in wild type mice) and lower naïve T cell numbers (17.7%) in comparison with wild type T cell population (28.6%) (FIG. 4B). The effect of TIRC7 deletion on costimulatory molecules on T cells was examined including CD40L, CTLA4, PD1, CD28 and ICOS as demonstrated in FIG. 4C-D.

CTLA4 molecule is described to be present at higher concentration in intracellular compartments compared to its cell surface expression; see Alegre, J. Immunol. 157 (1996), 4762-4770. Therefore, CTLA4 expression analysis was performed using permeabilized as well as non-permeabilized lymphocytes for FACS analysis. The intracellular and cell surface expression of CTLA4 was determined by FACS analysis 48 h after mitogenic activation of T cells. Strikingly, in activated T cells from TIRC7 (−/−) mice only minimal intracellular as well as cell surface expression of CTLA4 was detectable (FIG. 4C, a-c). In contrast, CTLA4 expression was unaffected in WT mice and was upregulated after 48 h activation. CD28 and ICOS expression levels were significantly decreased on TIRC7 (−/−) cells in contrast to WT littermates (FIG. 4D) whereas no significant changes in expression was observed for PD1 and CD40L in resting and activated T cells from TIRC7 mutant and wild type mice.

It is known that a number of cell surface molecules are being transported to cell surface via clathrin-coated vesicles. In order to examine whether TIRC7 deletion affects molecules known to be transported via clathrin-coated vesicles to cell surface such as CD71, lymphocytes were isolated from mutant and WT mice and activated with PHA. The expression of CD71 (FIG. 4E) was determined by FACS analysis. No significant differences in the expression of CD71 were observed between TIRC7 (−/−) cells and WT mice. These results strongly suggest that TIRC7 might deliver distinct signals regulating other signalling pathways of molecules known to be essential in immune response.

### Example 6

### Mice Lacking TIRC7 are More Susceptible to Antigen in vivo

Delayed-type hypersensitivity (DTH) is characteristically mediated by T cell response and Th-1 cytokines. Targeting of TIRC7 has been shown to affect the expression of these cytokines; see Utku, Immunity. 9 (1998), 509-518. Therefore the functional effects of TIRC7 deletion in mediation of inflammation and leukocyte recruitment was studied during an immune response in vivo by utilizing the DTH reaction. Mice were sensitised to ovalbumin by intradermal injection and seven days after immunization, the mice were challenged at day 8 and foot pad swelling was measured. DTH response to ovalbumin (Sigma) was estimated by foot pad swelling as previously described in Current Protocols in Immunology. Briefly, mice were sensitised with an injection of 50 μl of 5% (w/v) ovalbumin (ova) emulsified in Freunds Complete Adjuvant (Sigma) at the base of the tail. Eight days after the initial immunization, mice were challenged with an injection of 30 μl of 2% (w/v) ova in PBS into the left planar foot pad and 30 μl of PBS alone in the right planar foot pad. Foot pad thickness (swelling and erythema) were measured in both foot pads and the magnitude of the DTH reaction was determined as the difference in foot pad thickness between ova- and PBS-injected foot pads. Foot pad swelling was peaked 48 h after challenge which was significantly higher in TIRC7 knock out mice than observed in wild type littermates (FIG. 5A). As expected, assays of Th-1 cytokines revealed even higher levels of IFN-γ and IL-2 of TIRC7-deficient mice splenocytes stimulated 48 h with mitogen compared to WT littermates after the ova-challenge. Histology analysis of skin obtained form swollen footpads confirmed expected inflammation signs such as mononuclear infiltration of lymphocytes in WT animals, which was increased in TIRC7 deficient mice, as shown in FIG. 5B.

### Example 7

### Deletion of TIRC7 Results in Increased B Cell Activation and Elevated Immuno Globulin Levels

To further characterize TIRC7 (−/−) role on B cell activation we measured cell proliferation of splenocytes in vitro following 48 h incubation with various B cell stimuli including anti-CD40 antibody alone, or with LPS in combination with IL-4. For B-cell proliferation assays, lymphocytes (2×10⁶ cells/ml) were stimulated with 10 U/ml IL-4 and, either 0.5 μg/ml anti-CD40 mAb (Pharmingen) or 0.2 μg/ml LPS (Sigma), for 48 h. Cells were pulsed with 2 μCi [³H] thymidine and cell proliferation was measured after 16 h. Levels of IgM and IgG were measured in supernatants of 7 day old cultures by ELISA using capture-, detection- and standard-antibodies obtained from PharMingen, see example 2 and 4. As shown in FIG. 6A, in TIRC7 (−/−) splenocytes, in contrast to WT substantially higher levels of proliferation were observed following 48 h activation with all B cell stimuli. This was accompanied by increased levels of IgM and IgG1 production as compared to WT (FIG. 6B). Blood was obtained from the retro-orbital plexus of mice. The serum concentrations of IgM, IgG1, IgG2a, IgG2b, IgG3, IgA and IgE were determined by ELISA using capture-, detection- and standard-antibodies obtained from PharMingen. The measurement of immunoglobulin levels (Ig) in the serum by ELISA of mutant mice compared to WT showed increased levels of all immunoglobulin subclasses supporting the marked B cell activation in the mutant mice (FIG. 6C).

In order to analyse expression of costimulatory molecules on B cells splenocytes were incubated in the presence and absence of LPS and IL4 and surface expression of CD80 and CD86 was detected by flow cytometry. As shown in FIG. 6D CD86 is upregulated in resting B cells in knock out mice whereas no significant changes in CD80 expression was observed in knock out B cells in comparison to WT littermates, indicating that TIRC7 regulates distinct signalling pathways in B cells.

### Example 8

### Macrophages Revealed Morphological and Functional Defects in TIRC7 Deficient Mice

As shown in FIG. 7A, after 48h of stimulation with LPS and IL-4, TIRC(−/−) peritoneael macrophages showed significantly reduced number of proliferating cells (KO) compared with wild type (WT). The peritoneal cavity was washed with RPMI 1640 medium and the number of macrophages was determined with Neubauer hemocytometer. 1×10⁶ cells in RPMI 1640 medium supplemented with 10% fetal calf serum, 1 mM L-glutamine and streptomycin-penicillin were stimulated with the LPS (100 ng/ml) and recombinant IL-4 (10 ng/ml) at 37° C., 5% CO₂ for 48 h. The number of proliferating cells was quantified by microscopy. The phagocytosis analyzed by FACS revealed reduction of the overall percentage of macrophages and granulocytes showing phagocytosis cells in TIRC7 deficient cells compared with wild type. In order to analyze whether the TIRC7 deficiency affects the cytoskeleton which might lead to reduced ability of phagocytosis confocal microscopic analysis of TIRC7(−/−) macrophages were performed by using several specific antibodies against cytoskeleton molecules. Macrophages of the peritoneal cavity were coated 1 h at 37° C. on slides pretreated with Poly-L-Lysin (1:10, Sigma) and fixed after 20 min at 4° C. with 4% PFA. Cells were blocked with 5% milk for 1 h at room temperature and permeabilized with PBS/Triton (100× 0.5%) for 10 min at room temperature. Staining was performed using anti-actin (1:50, Santa Cruz), anti-tubulin (1:50, Santa Cruz) and anti-vinculin (1:50, Santa Cruz) rabbit polyclonal antibodies, or IgG rabbit control antibody (1:50, Santa Cruz) and incubation at 4° C. over night. The secondary antibody, cy3 labeled anti-rabbit antibody (1:250, Dianova) was incubated for 1 h at room temperature. Staining was analyzed using a Pascal 5 confocal microscope. As demonstrated in FIG. 7B, TIRC7 deficient macrophages exhibit expression defects off all cytoskeleton proteins tested (actin, tubulin and vinculin).

1. A composition of matter for treating therapeutically or prophylacticly a mammal afflicted with a disorder ameliorated by an increase in phagocytosis and/or monocyte population, which comprises a therapeutically effective amount of T-cell immune response cDNA 7 (TIRC7), an activator of TIRC7 or of a nucleic acid molecule encoding said TIRC7 or said activator, and optionally a pharmaceutically or cosmetically acceptable carrier.

2. A composition of matter for treating therapeutically or prophylacticly a mammal afflicted with a disorder ameliorated by a decrease in phagocytosis and/or monocyte population, which comprises a therapeutically effective amount of an antagonist of T-cell immune response cDNA 7 (TIRC7) or of a nucleic acid molecule encoding said antagonist, and optionally a pharmaceutically or cosmetically acceptable carrier.

3. The composition of claim 1, wherein TIRC7 is a recombinant TIRC7, a functional derivative thereof or a functionally equivalent substance.

4. The composition of claim 1, wherein the composition comprises a stimulatory anti-TIRC7 antibody, a TIRC7 ligand or a cell (over)expressing TIRC7.

5. The composition of claim 2, wherein the antagonist blocks an interaction of TIRC7 and its ligand.

6. The composition of claim 2, wherein the antagonist is or comprises an antibody, a (poly)peptide, a nucleic acid molecule, a TIRC7 gene targeting vector, a small organic compound, a TIRC7 ligand, peptide nucleic acid (PNA), aptamer, or peptide mimetic.

7. The composition of claim 6, wherein the antagonist is designed to be expressed in monocytes.

8. The composition of claim 2, wherein the antagonist comprises
(i) an anti-TIRC7 antibody or an anti-TIRC7-ligand antibody; or
(ii) a non-stimulatory form of TIRC7 or of its ligand.

9-10. (canceled)

11. A method of increasing phagocytosis and/or monocyte population, comprising
contacting a mammalian cell with an effective amount of T-cell immune response cDNA 7 (TIRC7), an activator of TIRC7 or of a nucleic acid molecule encoding said TIRC7 or said activator.

12. The method of claim 11, comprising p1 (a) obtaining cells, tissue or an organ from a subject;
(b) introducing into said cells, tissue or organ a nucleic acid molecule encoding and capable of expressing TIRC7 or its ligand in vivo; and
(c) reintroducing the cells, tissue or organ obtained in step (b) into the same subject or a different subject.

13. A method of decreasing phagocytosis and/or monocyte population, comprising contacting a mammalian cell with an effective amount of an antagonist of T-cell immune response cDNA 7 (TIRC7) or of a nucleic acid molecule encoding said antagonist.

14. A method of treating therapeutically or prophylacticly a mammal afflicted with a disorder ameliorated by an increase in phagocytosis and/or monocyte population, which comprises administering to the mammal a therapeutically effective amount of T-cell immune response cDNA 7 (TIRC7), an activator of TIRC7 or of a nucleic acid molecule encoding said TIRC7 or said activator.

15. A method of treating therapeutically or prophylacticly a mammal afflicted with a disorder ameliorated by a decrease in phagocytosis and/or monocyte population, which comprises administering to the mammal a therapeutically effective amount of an antagonist of T-cell immune response cDNA 7 (TIRC7) or of a nucleic acid molecule encoding said antagonist.

16. The method of claim 11, wherein the antagonist or activator is an agent.

17. An article of manufacture for administering to a mammal the composition of matter of claim 1, comprising a solid delivery vehicle having the composition operably affixed thereto.

18-22. (canceled)

23. A method to produce an immunoglobulin or an analog thereof, specific for a desired antigen, which method comprises:
(a) administering said antigen or an immunogenic portion thereof to a nonhuman animal under conditions to stimulate an immune response, whereby said animal produces B cells that secrete immunoglobulin specific for said antigen; wherein said nonhuman animal is characterized by being substantially incapable of producing endogenous T-cell immune response cDNA 7 (TIRC7) or TIRC7 activity in lymphocytes; and
(b) recovering said immunoglobulin or analog.

24. A method to produce an immunoglobulin or an analog thereof, specific for a desired antigen, which method comprises administering said antigen or an immunogenic portion thereof to a nonhuman animal under conditions to stimulate an immune response, whereby said animal produces B cells that secrete immunoglobulin specific for said antigen; wherein the endogenous T-cell immune response of said nonhuman animal is inhibited by administering an agent as defined in claim 2.

25. The method of claim 23, wherein the antigen or an immunogenic portion thereof is administered in conjunction with an agent.

26. The method of claim 23, further comprising recovering said polyclonal immunoglobulin or analog from said animal.

27. The method of claim 23, further comprising immortalizing B cells from said animal immunized with said antigen, screening the resulting immortalized cells for the secretion of said immunoglobulin specific for said antigen, and
(i) recovering immunoglobulin secreted by said immortalized B cells, or
(ii) recovering the genes encoding at least the immunoglobulin from the immortalized B cells, and optionally modifying said genes;
(iii)expressing said genes or modified forms thereof to produce the immunoglobulin or analog; and
(iv) recovering said immunoglobulin or analog.

28. The method of claim 23, further comprising
(i) recovering genes encoding the immunoglobulins from the primary B cells of the animal;
(ii) generating a library of said genes expressing the immunoglobulins;
(iii)screening the library for an immunoglobulin with the desired affinity for the antigen;
(iv) recovering the genes encoding the immunoglobulin;
(v) expressing said genes to produce an immunoglobulin or analog;
(vi) recovering said immunoglobulin or analog.

29. The method of claim 23, wherein the desired antigen is selected from the group consisting of transition state mimics; leukocyte markers; histocompatibility antigens; adhesion molecules; interleukins; interleukin receptors; chemokines; growth factors and their receptors; interferon receptors; Igs and their receptors; tumor antigens; allergens; viral proteins; toxins; blood factors; enzymes; ganglioside GD3, ganglioside GM2, LMP1, LMP2, eosinophil major basic or cationic protein, pANCA, Amadori protein, Type IV collagen, glycated lipids, γ-interferon, A7, P-glycoprotein, Fas (AFO-1) and oxidized-LDL; human IL-6 or IL-8, human TNFα, human CD4, human L-selectin, human gp39, human IgE, human αVβ3, human Fibrinosin (FₛF₋₁), human laminin, human PTHrP, and tetanus toxin C (TTC).

30-32. (canceled)

33. The method of claim 23, wherein said immunoglobulin or analog is an antibody or analog thereof.

34. The method of claim 23, further comprising the step(s) of producing a chimeric antibody, humanized antibody, single-chain antibody, Fab-fragment, bi-specific antibody, fusion antibody, labelled antibody or an analog of any one of those.

35. (canceled)

36. A vaccine comprising an agent as defined in claim 2.

37. (canceled)
